(19) United States
(12) Patent Application Publication
(10) Pub. No.: US 2004/0077537 A1
(43) Pub. Date: Apr. 22, 2004

(54) Method for treating congestive heart failure

(76) Inventor: George F. Schreiner, Los Altos, CA (US)

Correspondence Address: Scios Inc., 6500 Paseo Padre Parkway, Fremont, CA, 94555, US

(21) Appl. No.: 10/390,546

(22) Filed: Mar. 18, 2003

Related U.S. Application Data
(60) Provisional application No. 60/364,736, filed on Mar. 18, 2002.

(51) Int. Cl. A61K 38/17

(52) U.S. Cl. 514/12

(57) ABSTRACT

The present invention relates to methods for administration of natriuretic peptide that are especially useful for treatment of a CHF patient who is relatively compensated, or to a CHF patient in an outpatient setting, and especially as an adjunct to oral therapy. The methods are characterized by administration of a composition that provides a dose of natriuretic peptide that is lower than that dose typically administered to a CHF patient who is in need of acute treatment. The methods of the invention are also useful for the treatment of chronic CHF patients, especially subacute chronic CHF patients.

### BACKGROUND OF THE INVENTION

### FIELD OF THE INVENTION

The invention is in the field of natriuretic peptide and methods for administration of the same. The methods are especially useful for treating congestive heart failure patients in an outpatient setting.

### BACKGROUND OF THE INVENTION

Advanced congestive heart failure (CHF) accounts for over 1 million hospital admissions yearly in the United States (U.S.) and is associated with a 5-year mortality rate of 40%-50%. (American Heart Association. 2001 Heart and Stroke Statistical Update. Dallas, Tex.: American Heart Association, 2000; Massie, B. M., and Shah, N. B., Am. Heart J. 133:703-712 (1997)) In the U.S., CHF is currently the most costly cardiovascular disease, with the total estimated direct and indirect costs approaching $56 billion in 1999. (Rich, M. W., and Nease, R. F., Arch Intern Med 159:1690-1700 (1999)) The estimated cost of a single hospital admission primarily for CHF is $11,000 (Massie, B. M., and Shah, N. B., Am. Heart J. 133:703-712 (1997); Rich, M. W., and Nease, R. F., Arch Intern Med 159:1690-1700 (1999)), whereas the Medicare Diagnostic-Related Group (DRG) reimbursement is approximately $4,300. This represents an increased economic burden for hospitals, as Medicare is the primary provider for this disease.

Efforts to contain rising costs for hospital acute care have resulted in shorter hospital stays for patients with acutely decompensated CHF. These shorter stays may not allow for adequate diuresis or titration of oral medications for patients to receive the full benefit of intravenously (IV) administered medications.

Additionally, there is an unmet need for effective outpatient treatment of advanced chronic CHF. Therapies that improve quality of life and/or reduce the number of hospital admissions for CHF are needed. While there is increasing interest among clinicians in the use of outpatient IV therapy for the treatment of advanced CHF, there is no consensus among clinicians regarding patient selection, dosing or treatment duration. The use of milrinone and dobutamine in this setting is controversial and may lead to increased mortality. (O'Connell, J. B., Clin. Cardiol. 23:III6-10 (2000)).

### BRIEF SUMMARY OF THE INVENTION

The present invention relates to methods for administration of natriuretic peptide. The methods of the invention are especially useful for treatment of a CHF patient who is relatively compensated, or to a CHF patient in an outpatient setting, and especially as an adjunct to oral therapy. The methods of the invention are characterized by administration of a composition that provides a dose of natriuretic peptide that is preferably lower than that dose typically administered to a CHF patient who is in need of acute treatment. The methods of the invention are also useful for the treatment of chronic CHF patients, especially subacute chronic CHF patients.

Accordingly, in a first embodiment, the invention is directed to a method for the administration of natriuretic peptide to a patient who has been diagnosed as having advanced congestive heart failure and who is in a relatively compensated state but in need of management of a risk of heart failure, such method comprising administration of a therapeutically effective dose of natriuretic peptide to said patient, such therapeutically effective dose preferably being in adjunct to oral therapy and at a dose that is lower than the dose used to treat acute CHF.

In a further embodiment, the such natriuretic peptide is atrial natriuretic peptide (ANP) or more preferably, B-type natriuretic peptide (BNP).

In a further embodiment, natriuretic peptide is human natriuretic peptide.

In a further embodiment, the such human natriuretic peptide is recombinant human B-type natriuretic peptide (nesiritide).

In a further embodiment, such administration is in the form of an infusion, preferably an intravenous infusion, and most preferably a serial intravenous infusion.

In a further embodiment, the composition of the invention is provided as the primary IV vasoactive therapy for a patient with CHF, and such patient is infused with therapeutically effective doses of natriuretic peptide for 4-6 hours weekly.

### BRIEF DESCRIPTION OF THE FIGURES

FIG. 1. A schematic representation of the amino acid sequence of human B-type natriuretic peptide (SEQ ID NO:1).

FIG. 2. A graph of pulmonary capillary wedge pressure (PCWP) in patients in the VMAC (Vasodilation in the Management of Acute Congestive Heart Failure) study.

FIG. 3. Mean hemodynamic change from baseline in patients in the VMAC (Vasodilation in the Management of Acute Congestive Heart Failure) study.

FIG. 4. Adverse reactions in patients in the VMAC study.

FIG. 5. Calculation of the appropriate bolus volume and infusion flow rate for conventional administration of Natrecor®.

### DETAILED DESCRIPTION OF THE INVENTION

### Definitions

The term “ameliorate” denotes a lessening of an effect. To ameliorate a condition or disease refers to a lessening of the symptoms of the condition or disease.

An “individual” is a vertebrate, preferably a mammal, more preferably a human.

“Mammal” refers to any animal classified as a mammal, including humans, domestic and farm animals, and zoo, sport, or pet animals, such as, for example, horses, sheep, cows, pigs, dogs, cats, etc. Preferably, the mammal is human.

A “therapeutically effective amount” or an “effective amount” is an amount sufficient to effect beneficial or desired results. An effective amount can be provided in one dose. Alternatively, the effective amount can be provided in multiple doses for a desired period of time, such multiple doses being cumulatively sufficient to effect the beneficial or desired result but each of such multiple doses being at an individual level that may or may not be effective had it been administered by itself.

“Administration” means any manner of providing a desired agent to a subject or patient. Administration “in combination with” one or more further therapeutic agents means any manner with provides for the beneficial effects of the administration of both agents, including simultaneous (concurrent) administration and consecutive administration in any order.

The term “modulate” means to control in a predictable fashion, either by increasing or by decreasing the targeted parameter, as indicated from the context.

A “treatment” is an approach for obtaining a beneficial or desired result, especially a clinical result, especially the administration of an agent to a subject for purposes which may include prophylaxis, amelioration, prevention or cure of an undesired physiological condition or disease. Such treatment need not necessarily completely ameliorate the condition or disorder. For purposes of this invention, beneficial or desired clinical results include, but are not limited to, alleviation of symptoms, diminishment of extent of tissue injury or disease, stabilized (i.e., not worsening) state of tissue injury or disease, delay or slowing of the progression or tissue injury or disease, amelioration or palliation of an undesired physiological condition or disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented.

“Carriers” as used herein include pharmaceutically acceptable carriers, excipients, or stabilizers which are nontoxic to the cell or mammal being exposed thereto at the dosages and concentrations employed.

The term “pharmaceutically acceptable salt” as used herein refers to salt forms of a substance that are substantially non-toxic to living organisms. Typical pharmaceutically acceptable salts include those salts prepared by reaction of a desired agent, such as a desired form of natriuretic peptide, with a pharmaceutically acceptable mineral or organic acid or an inorganic base. Such salts are known as acid addition and base addition salts.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of pharmaceutically acceptable salts formed from such acids are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma.-hydroxybutyrate, glycollate, tartrate, methanesulfonate, propanesulfonate, naphthalene-1-sulfonate, naphthalene-2-sulfonate, mandelate, mesylate, and the like. Preferred pharmaceutically acceptable acid addition salts are those formed with mineral acids such as hydrochloric acid and hydrobromic acid, and those formed with organic acids such as maleic acid and methanesulfonic acid.

Salts of amine groups may also comprise quaternary ammonium salts in which the amino nitrogen carries a suitable organic group such as an alkyl, alkenyl, alkynyl, or aralkyl moiety.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is not of a critical nature, so long as the salt as a whole is pharmacologically acceptable and as long as the counterion does not contribute undesired qualities to the salt as a whole.

The term “essentially free of contaminants” refers to a substance that is purified to a degree such that the substance contains no, or acceptable levels of, undesired or unnecessary substances that arose form, or had been present during, the in vitro or in vivo synthesis of the desired substance.

### Congestive Heart Failure

Congestive heart failure (CHF; cardiac failure) is a condition in which weakened heart function exists together with a build-up of body fluid. Cardiac failure often occurs when cardiac output is insufficient to meet metabolic demands of the body, or when the heart cannot meet the demands of operating at increased levels of filling/diastolic pressure. Therapy involves not only support of the weakened heart function but also treatment to counteract the build up of the body fluid.

Congestive heart failure may be caused by many forms of heart disease. Common causes of congestive heart failure include: narrowing of the arteries supplying blood to the heart muscle (coronary heart disease); prior heart attack (myocardial infarction) resulting in scar tissue large enough to interfere with normal function of the heart; high blood pressure; heart valve disease due to past rheumatic fever or an abnormality present at birth; primary disease of the heart muscle itself (cardiomyopathy); defects in the heart present at birth (congenital heart disease) and infection of the heart valves and/or muscle itself (endocarditis and/or myocarditis). Each of these disease processes can lead to congestive heart failure by reducing the strength of the heart muscle contraction, by limiting the ability of the heart's pumping chambers to fill with blood due to mechanical problems or impaired diastolic relaxation, or by filling the heart's chambers with too much blood.

Advanced congestive heart failure (CHF) includes both acute and chronic presentations. Patients presenting with acute decompensated CHF usually have an acute injury to the heart, such as a myocardial infarction, mitral regurgitation or ventricular septal rupture. Typically, the injury compromises myocardial performance (for example, a myocardial infarction) or valvular/chamber integrity (for example, mitral regurgitation or ventricular septal rupture). Such injuries result in an acute rise in the left ventricular (LV) filing pressures. The rise in the LV filing pressures results in pulmonary edema and dyspnea. The treatment of patients with acute decompensated CHF focuses on treating the reason behind the myocardial injury. In addition, the heart's function is supported by treatments to reduce LV filling pressures and to improve cardiac performance.

Patients with chronic decompensated heart failure often have symptoms of volume overload and/or low cardiac output—but do not appear to be in a volume overloaded state. Such patients thus have a chronic LV systolic dysfunction.

### The Compositions of the Invention

The invention provides pharmaceutically active compositions that are useful for both the prophylactic and therapeutic treatment of CHF for patients that are relatively compensated.. The pharmaceutically active compositions of the invention invention are characterized in containing a natriuretic peptide, sufficient to provide a therapeutically effective amount of a natriurertic peptide to such patient when administered in an appropriate dose and for an appropriate period of time.

The natriuretic peptide that is present in a composition of the invention can be any of the family of therapeutically effective natriuretic peptides, or a mixture of the same. Examples of useful natriuretic peptides include, for example, atrial natriuretic peptide (ANP), brain natriuretic peptide (BNP or B-type natriuretic peptide) and C-type natriuretic peptide (CNP). Of them, ANP and BNP are preferred, and BNP is the most preferred. Sequences of many useful forms of natriuretic peptide are provided in (U.S. patent application Publication Ser. No. 10,027,181A1, 2001 incorporated by reference herein).

Specific examples of ANPs that can be used in the methods of the invention include: human ANP (human atrial natriuretic peptide; hANP, Kangawa et al., Biochem. Biophys. Res. Commun., Vol.118, p.131, 1984) (Seq. ID No. 1) or rat ANP (Kangawa et al., Biochem. Biophys. Res. Commun., Vol. 121, p. 585, 1984). Such ANPs comprise 28 amino acids. Such ANPs may be administered as a peptide having a ring structure of ANP (formation of a disulfide bond based on Cys), and a C-terminal portion succeeding the ring structure. An example of such a peptide is a peptide having amino acid residues at the 7-position to the 28-position of ANP is provided in U.S. patent application Publication Ser. No. 0,027,181 A1. 2000 Another example is frog ANP. Of them, human ANP (hANP), and especially recombinant hANP is particularly preferred.

Specific examples of BNPs that can be used in the methods of the invention include human BNP (hBNP). Human BNP comprises 32 amino acids and involves the formation of a disulfide bond, like the above-described ANP (Sudoh et al., Biochem. Biophys. Res. Commun., Vol. 159, p. 1420, 1989). See also, U.S. Pat. Nos. 5,114,923, 5,674,710, 5,674,710, 5,948,761, each of which is hereby incorporated by reference. Various BNP's of the origin other than human, such as pig BNP and rat BNP, are also known, and can be used similarly. A further example is chicken BNP.

Specific examples of CNPs that can be used in the methods of the invention include pig CNP. Pig CNP comprises 22 amino acids and involves the formation of a disulfide bond, like the above-described ANP and BNP (Sudoh et al., Biochem. Biophys. Res. Commun., Vol. 168, p. 863, 1990) (human and rat also have the same amino acid sequence), chicken CNP (Arimura et al., Biochem. Biophys. Res. Commun., Vol. 174, p. 142, 1991). Frog CNP (Yoshihara et al., Biochem. Biophys. Res. Commun., Vol. 173, p. 591, 1990) can also be used.

Furthermore, any person skilled in the art can apply modification, such as deletion, substitution, addition or insertion, and/or chemical modification to amino acid residues in the amino acid sequence of a known natriuretic peptide (e.g., the aforementioned human ANP; hANP), as desired, by a known method. One skilled in the art can confirm that the resulting compound is a compound which has the activity of acting on a receptor of the starting ANP or BNP or CNP. Derivatives having this activity, therefore, are included in the substance as an active ingredient which is administered to a patient in accordance with the method of the present invention.

A substance that activates the patient's natriuretic peptide receptor could also be uses in the compositions of the invention in place of, or in addition to, one or more of the natriuretic peptides discussed above. Such substance should be capable of acting on a natriuretic peptide receptor to increase intracellular cGMP production. Such substances may be non-peptide compounds.

In the compositions of the invention, the natriuretic peptide is preferably provided as a free (non-salt) form, or as a pharmaceutically acceptable salt. A salt with an inorganic acid preferably includes salts with hydrochloric acid, sulfuric acid, and phosphoric acid. The salt with an organic acid thus may, preferably be, for example, acid addition salts with formic acid, acetic acid, butyric acid, succinic acid, and citric acid. The salt is preferably in the form of a metal salt with sodium, potassium, lithium or calcium, or a salt with an organic base.

To produce compositions for infusion, carriers or additives can be added to provide a desired stability or property to the composition. Examples of such carriers and additives include: (1) tonicity agents such as sodium chloride, D-mannitol, and D-sorbitol, (2) pH regulators such as hydrochloric acid and citric acid, (3) buffering agents such as sodium citrate, sodium acetate, and boric acid, and (4) soothing agents such as procaine hydrochloride; as well as stabilizers, and surface active agents. Often the physiologically acceptable carrier is an aqueous pH buffered solution. Examples of physiologically acceptable carriers include buffers composed of phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

In consideration of the stability, etc. of the active natriuretic peptide ingredient, it can be selected whether the active ingredient should be formed into a preparation to be used after dissolution or suspension when required, or into a liquid preparation.

### The Methods of the Invention

According to the invention, serial and intermittent administration of natriuretic peptides provides therapeutic and prophylactic benefit to CHF patients. In a preferred embodiment, CHF patients who are in a relatively stable or compensated state are administered a bolus of natriuretic peptide coupled with serial and intermittent infusion to achieve the desired treatment. By “relatively stable or relatively compensated,” it is meant that the CHF patient, at the time of administration of a therapeutic and/or prophylactic dosage of a natriuretic peptide according to the present invention, is not exhibiting acute heart failure symptoms necessitating immediate treatment. Such patients may include, for example, patients in New York Heart Association functional classifications I through IV as described in Table 2, as well as patients who may have recently been hospitalized for previous treatment of acute heart failure. Preferably, the peptide dosage can range to where the bolus is about, not less than 2 μg/kg and the serial and intermittent infusion is less than about 0.01 μg/kg/min.

The pharmaceutically active compositions that provide the active natriuretic peptide are preferably administered to the patient who is in need of the same in the form of an injection, Such injections can be, for example, intravenous, intramuscular, subcutaneous, intradermal, intrasternal, intraperitoneal or intra-articular. Most preferably, the compositions are provided in the form of an infusion, and especially, an intravenous infusion.

In a preferred embodiment, therapeutically effective doses of natriuretic peptides and especially, nesiritide (commercially sold as Natrecor®) are used in the compositions of the invention in a form intended for injection or infusion. Preferably, such compositions reduce pulmonary capillary wedge pressure and to improve patients with dyspnea at rest or with minimal activity.

The infusion can be administered for any effective period of time, for example, 1, 2, 3, 4, 5, 6, 7, 8, 9 or 10 hours, or a desired period of time in between. In a further embodiment, infusions for greater than 10 hours are performed. In a preferred embodiment, a patient in need of such treatment is infused for 4-6 hours. Preferably such infusion is continuous although two infusions of shorter duration following one right after the other may be used.

The infusion rate may be any that is tolerated by the patient. Examples of useful bolus amounts and infusion rates are provided in Table 1.

Higher or lower infusion rates can be used if desired. In a preferred embodiment, the infusion rate is about 0.00125 μg/kg/min to about 0.01 μg/kg/min. In a further preferred embodiment, an infusion rate of about 0.005 μg/kg/min is used.

The infusion rate should be sufficient to provide a therapeutically effective amount of the natriuretic peptide during the infusion period or treatment protocol but without compromising patient safety.

If one embodiment, a separate initial bolus of a preparation that contains natriuretic peptide is administered to the patient immediately prior to a subsequent infusion. Such a bolus preferably provides from about 0.25, 0.5, 0.75, 1.0, 1.25, 1.5 or 1.75 μg/kg natriuretic peptide.

The optimal volume of the infusion and amount of the active natriuretic peptide will vary by body weight. For example, a 30 kg (66 pound) patient might first be given a 2.5 ml bolus of 0.5 μg/ml of the natriuretic peptide composition infused at 0.8 ml/hr so as to provide for 0.0025 μg/kg/min, followed by a minimum bolus of 0.25 μg/kg in 1.3 mL infused at 0.4 mL/hr and 0.00125 μg/kg/min to a maximum bolus of 1.0 μg/kg in 5.0 mL infused at a maximal rate of 1.5 mL/hr to provide 0.005 μg/kg/min.

For another example, a 30 kg (66 pound) patient might first be given a 5.0 ml bolus of 1.0 μg/ml of the natriuretic peptide composition infused at 1.5 ml/hr so as to provide for 0.005 μg/kg/min, followed by a minimum bolus of 0.5 μg/kg in 2.5 mL infused at 0.8 mL/hr and 0.0024 μg/kg/min.

In contrast, a 175 kg (386 pound) patient might first be given a 14.6 ml bolus of 0.5 μg/ml of the natriuretic peptide composition infused at 4.4 ml/hr so as to provide for 0.0025 μg/kg/min, followed by a minimum bolus of 0.25 μg/kg in 7.3 mL infused at 2.2 mL/hr and 0.00124 μg/kg/min to a maximum bolus of 1.0 μg/kg in 29.2 mL infused at a maximal rate of 8.8 mL/hr to provide 0.005 μg/kg/min.

Alternatively, a 175 kg (386 pound) patient might first be given a 29.2 ml bolus of 1.0 μg/ml of the natriuretic peptide composition infused at 8.8 ml/hr so as to provide for 0.005 μg/kg/min, followed by a minimum bolus of 0.5 μg/kg in 14.6 mL infused at 4.4 mL/hr and 0.0025 μg/kg/min. Additional examples are provided in Table 1.

In a preferred embodiment, the methods of the invention provide therapeutically effective doses of natriuretic peptides, and especially, nesiritide, to a patient in need of long term management and/or at high risk of heart failure. Such treatment preferably occurs in a subacute or outpatient setting. Infusion of the composition of the invention is therapeutically effective if it provides a desirable hemodynamic and neurohormonal effects in addition to maintaining the advantageous safety profile of the administered natriuretic peptide.

The method of the invention is especially useful as an add-on therapy to oral medications. In a highly preferred embodiment of the method of the invention, serial IV infusions of a netriuretic peptide, and especially of nesiritide are provided at therapeutically effective doses to patients who are also being treated with oral therapeutic agents to manage such patient's CHF, such therapeutically effective doses of the infused natriuretic peptide resulting at least in part in a more rapid and sustained compensation of CHF in patients with frequent episodes of acutely decompensated CHF, and/or who are in need of chronic CHF treatment in a subacute or outpatient setting.

It is an advantage of the invention that the method of the invention can be used to treat patients who are in a relatively compensated state. According to the invention, the appropriate dose of natriuretic peptide, and especially, the appropriate dose of nesiritide, is provided by infusion and at a lower dose than that generally used in an acutely decompensated setting and maybe better tolerated by the patient in need of treatment.

Having now generally described the invention, the same will become better understood by reference to certain specific examples which are included herein for purposes of illustration only and are not intended to be limiting unless other wise specified. All referenced publications and patents are incorporated, in their entirety by reference herein.

### EXAMPLES

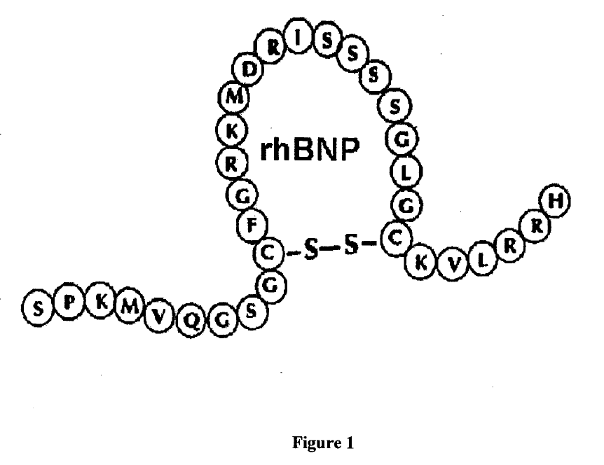

The following abbreviations are used:

| | |
|---|---|
| ACE | angiotensin converting enzyme |
| AICD | automatic implantable cardiac defibrillator |
| ANOVA | analysis of variance |
| ARB | angiotensin receptor blocker |
| BP | blood pressure |
| BNP | B-type natriuretic peptide |
| CI | cardiac index |
| CHF | congestive heart failure |
| CRF | case report form |
| DRG | diagnosis related group |
| EF | ejection fraction |
| EKG | electrocardiogram |
| hBNP | human B-type natriuretic peptide (FIG. 1) |
| HR | heart rate |
| IV | intravenous |
| LVEDI | left ventricular end diastolic index |
| NYHA | New York Heart Association |
| MLwHF | Minnesota Living with Heart Failure Questionnaire |
| PE | physical examination |
| QoL | Quality of Life |
| RR | respiratory rate |
| SBP | systolic blood pressure |
| SVI | stroke volume index |
| SVR | systemic vascular resistance |
| VS | vital signs |
| Wt | weight |

### Example 1

The exemplified embodiments utilize Natrecor® as the natriuretic peptide. Natrecor® is a proprietary name for a recombinant form of human B-type natriuretic peptide (hBNP), also known as brain natriuretic peptide. It is identical to the endogenous hormone that is produced primarily by the ventricular myocardium. (Ewy, G. A., J. Am. Coll. Cardiol. 33(2):572-575 (1999)) However, it is to be understood that the examples are not restricted to Natrecor®. The exemplified embodiments provide an outline for the establishment of the use of any natriuretic peptide in the methods and treatments of the invention.

IV administration of Natrecor® produces balanced vasodilation, antagonism of the renin-aldosterone system, and diuresis. (Hosoda, K., et al., Hypertension 17:1152-1155 (1991); Marcus, L. S., et al. Circulation 94:3184-3189 (1996)) When administered to CHF patients for up to 24 hours, a continuous infusion of Natrecor® resulted in significant dose related reductions in cardiac filling pressures and systemic vascular resistance (SVR), with increases in cardiac index (CI) and no change in heart rate (HR). (Abraham, W. T., et al., J. Am. Coll. Cardiol. 236A (1995)) In a6-hour placebo controlled comparison in patients with acutely decompensated CHF, Natrecor® was also associated with significant improvement in the symptoms of CHF (including dyspnea and fatigue), a decrease in aldosterone, and an increase in urine output. (Mills, R. M., et al., J. Am. Coll. Cardiol. 34(1):155-62 (1999)).

Natrecor® generally has been well tolerated in controlled clinical trials involving more than 1000 patients with CHF.

### Description

Natrecor* (nesiritide) is a sterile, purified preparation of a now drug class, human B-type natriuretic peptide (hBNP), and is manufactured from E. coli using recombinant DNA technology. Nesiritide has a molecular weight of 3464 g/mol and an empirical formula of C₁₄₃H₂₄₄N₅₀O₄₂S₄. Nesiritides has the same 32 amino acid sequence as the endogenous peptide, which is produced by the ventricular myocardium.

Natrecor is formulated as the citrate salt of rhBNP, and is provided in a sterile, single-use vial. Each 1.5 mg vial contains a white-to off-white lyophilized powder for intravenous (IV) administration after reconstitution. The quantitative composition of the lyophilized drug per vial is: nesiritide 1.58 mg, mannitol 20.0 mg, citric acid monohydrate 11 mg, and sodium citrate dihydrate 2.94 mg.

### Mechanism at Action

Human BNP binds to the particulate guanylate cyclase receptor of vascular smooth muscle and endothelial cells, leading to increased intracellular concentrations of guanosine 3′5′-cyclic monophosphate (cGMP) and smooth muscle cell relaxation. Cyclic GMP serves as a second messenger to dilate veins and arteries. Nesiritide has been shown to relax isolated human arterial and venous tissue preparations that were precontracted with either endothelin-1 or the alpha-adrenergic agonist, phenylephrine.

In human studies, nesiritide produced dose-dependent reductions in pulmonary capillary wedge pressure (PCWP) and systemic arterial pressure in patients with heart failure.

In animals, nesiritide had no effects an cardiac contractility or on measures of cardiac electrophysiology such as atrial and ventricular effective refractory times or atrioventricular node conduction.

Naturally occurring atrial natriuretic peptide (ANP), a related peptide, increases vascular permeability in animals and humors and may reduce intravascular volume. The effect of nesiritide on vascular permeability has not been studied.

### Pharmacokinetics

In patients with congestive heart failure (CHF), Natrecor administered intravenously by infusion or bolus exhibits biphasic disposition from the plasma. The mean terminal elimination half-life (t_{1/2}) of Natrecor is approximately 10 minutes and was associated with approximately ⅔ of the area-under-the-curve (AUC). The mean initial elimination phase was estimated to be approximately 2 minutes. In these patients, the mean volume of distribution of the control compartment (Vc) of Natrecor was estimated to be 0.073 Mg, the mean steady-state volume of distribution (Vss) was 0.19 L/kg, and the mean clearance (CL) was approximately 9.2 mL/min/kg. At steady state, plasma BNP levels increase from baseline endogenous levels by approximatly 3-fold to 6-fold with Natrecor infusion doses ranging from 0.01 to 0.03 μg/kg/min.

### Elimination

Human BNP is cleared from the circulation via the following three independent mechanisms, in order of decreasing importance: 1) binding to cell surface clearance receptors with subsequent cellular Internalization and lysosomal proteolysis; 2) proteolytic cleavage of the peptide by endopeptidases, such as neutral endopeptidase, which are present on the vascular lumenal surface; and 3) renal filtration.

### Special Populations

Although Natrecor is eliminated, in part, through renal clearance, clinical date suggest that dose adjustment is not required in patients with renal insufficiency. The effects of Natrecor on PCWP, cardiac index (CI), and systolic blood pressure (SBP) were not significantly different in patients with chronic renal insufficiency (baseline serum creatinine ranging from 2 mg/dL to 4.3 mg/dl), and patients with normal renal function. The population pharmacokinetic (PK) analyses carried out to determine the effects of demographics and clinical variables on PK parameters showed that clearance of Natrecor is proportional to body weight supporting the administration of weight-adjusted dosing of Natrecor (i.e., administration on a μg/kg/min basis). Clearance was not influenced significantly by age, gander, race/ethnicity, baseline endogenous hBNP concentration, severity of CHF (as indicated by baseline PCWP, baseline CI, or New York Heart Association [NYHA] classification) or concomitant administration of an ACE inhibitor.

### Effects of Concomitant Medications

The co-administration of Natrecor with enalapril did not have significant effects on the PK of Natrecor. The PK effect of co-administration of Natrecor with other IV vasodilators such as nitroglycerin, nitroprusside, milninone, or IV ACE inhibitors; has not been evaluated. During clinical studies, Natrecor was administered concomitantly with other medications, including: diuretics, digoxin, oral ACE inhibitors, anticoagulant, oral nitrates, statins, class III antiarrhythmic agents, beta-blockers, dobutamine, calcium channel blockers, angiotensin II receptor antagonists, and dopamine. Although no PK interactions were specifically assessed, there did not appear to be evidence suggesting any clinically significant PK interaction.

### Pharmacodynamics

The recommended dosing regimen of Natrecor is a 2 μg/kg IV bolus followed by an intravenous infusion dose of 0.01 μg/kg/min. With this dosing regimen, 60% of the 3-hour off act on PCWP reduction is achieved within 15 minutes after the bolus, reaching 95% of the 3-hour effect within 1 hour. Approximately seventy percent of the 3-hour effect on SBP reduction is reached within 15 minutes. The pharmacodynamic (PD) half-life of the onset end offset of the hemodynamic effect of Natrecor is longer than what the PK half-life of 18 minutes would predict. For example, in patients who developed symptomatic hypotension in the VMAC (Vasodilation in the Management of Acute Congestive Heart Failure) trial, bag of the recovery of SBP toward the baseline value after discontinuation or reduction of the dose of Natrecor was observed in about 60 minutes. When higher doses of Natrecor were infused, the duration of hypotension was sometimes several hours.

### Clinical Trials

Natrecor has been studied in 10,clinical trials including 941 patients with CHF (NYHA class II-III 61%, NYHA class IV 36%; mean age 60 years, women 28%). There were five randomized, multi-center, placebo- or active-controlled studies (comparative agents included nitroglycerin, dobutamine, milrinone, nitroprusside, or dopamine) in which 772 patients with decompensated CHF received continuous infusions of Natrecor at doses ranging from 0.01 to 0.03 μg/kg/min. (See the ADVERSE REACTION section for relative frequency of adverse events at doses ranging from the recommended dose up to 0.03 μg/kg/min. Of these patients, the majority (n=541, 70%) received the Natrecor infusion for at least 24 hours; 371 (48%) received Natrecor for 24-48 hours, and 170 (22%) received Natrecor for greater then 48 hours.

In controlled trials, Natrecor has been used alone or in conjunction with other standard therapies, including diuretics (79%), digoxin (62%), oral ACE inhibitors (55%), anticoagulants (36%), oral nitrates (32%), statins (18%), class III antiarrhythmic agents (16%), beta-blockers (15%), dobutamine (15%), calcium channel blockers (11%), angiotensin II receptor antagonists (6%), and dopamine (4%). Natrecor has been studied in a broad range of patients, including the elderly (42%>65 years of age), women (30%), minorities (26% black), and patients with a history of significant morbidities such as hypertension (67%), previous myocardial infarction (50%), diabetes (44%), atrial fibrillation/flutter (34%), nonsustained ventricular tachycardia (25%) ventricular tachycardia/fibrillation (12%), preserved systolic function (9%), and acute coronary syndromes less than 7 days before the start of Natrecor (4%).

The VMAC (Vasodilation in the Management ofAcute, Congestive Heart Failure) trial was a randomized, double-blind study of 489 patients (246 patients requilring a right heart catheter, 243 patients without a right heart catheter) who required hospitalization for management at shortness of breath at rest due to acutely decompensated CHF. TN study, compared the effects of Natrecor, placebo, and IV nitroglycerin when added to background therapy (IV and oral diuretics, non-IV cardiac medications, dobutamine, and dopamine). Patients with acute coronary syndrome, preserved systolic function, arrhythmia, and renal insufficiency wore not excluded. The primary endpoints of the study were the change from baseline in PCWP and the change from baseline in patients' dyspnea, evaluated after three hours. Close attention was also paid to the occurrence and persistence of hypotension, given nesiritide's relatively long (compared to nitroglycerin) PK and PD half-life.

Natrecor was administered as a 2 μg/kg bolus over approximately 60 seconds, followed by a continuous fixed dose infusion of 0.01 μg/kg/min. After the 3-hour placebo-controlled period, patients receiving placebo crossed over to double-bladed active therapy with either Natrecor or nitroglycerin. The nitroglycerin dose was titrated at the physician's discretion. A subset of patients in the VMAC trial with central hemodynamic monitoring who were treated with Natrecor (62 of 124 patients) were allowed dose increases of Natrecor after the first 3 hours of treatment it the PCWP was ≧20 mm Hg and the SBP was ≧100 mm Hg. Dose increases of a 1 μg/kg bolus followed by an increase of the infusion dose by 0.005 μg/kg/min were allowed every 3 hours, up to a maximum dose of 0.03 μg/kg/min. Overall, 23 patients in this subset had the dose of Natrecor increased in the VMAC trial.

In a second double-blind study, 127 patients requiring hospitalization (of symptomatic CHF were randomized to placebo or to one of two doses of Natrecor (0.015 μg/kg/min preceded by an IV bolus of 0.3 μg/kg and 0.03 μg/kg/min preceded by an IV bolus of 0.6 μg/kg). The primary endpoint of the trial was the change in PCWP from baseline to 6 hours, bid the effect on symptoms also was examined.

### Effects an Symptoms

In the VMAC study, patients receiving Natrecor reported greater improvement in their dyspnea at 3 hours than patients receiving placebo (p=0.034).

In the dose-response study, patients receiving both doses of Natrecor reported greater improvement in dyspnea at 6 hours than patients receiving placebo.

### Effects on Hemodynamics

The PCWP, right atrial pressure (RAP), CI, and other hemodynamic variables were monitored in 246 of the patients in the VMAC trial. There was a reduction in mean PCWP within 15 minutes of starting the Natrecor infusion, with most of the effect seen at 3 hours being achieved within the first 60 minutes of the infusion (see Pharmacodynamics).

In several studies, hemodynamic parameters wars measured after Natrecor withdrawal. Following discontinuation of Natrecor, PCWP returns to within 10% of baseline within 2 hours, but no rebound increase to levels above baseline state was observed. There was also no evidence of tachyphylaxis to the hemodynamic effects of Natrecor in the clinical trials.

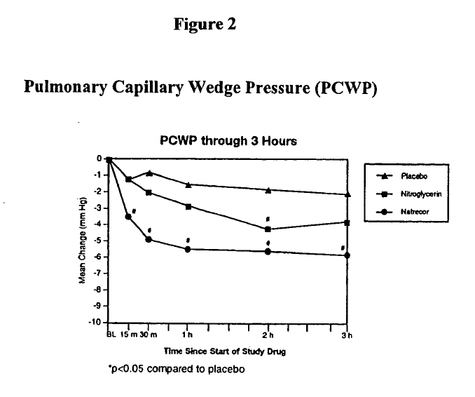

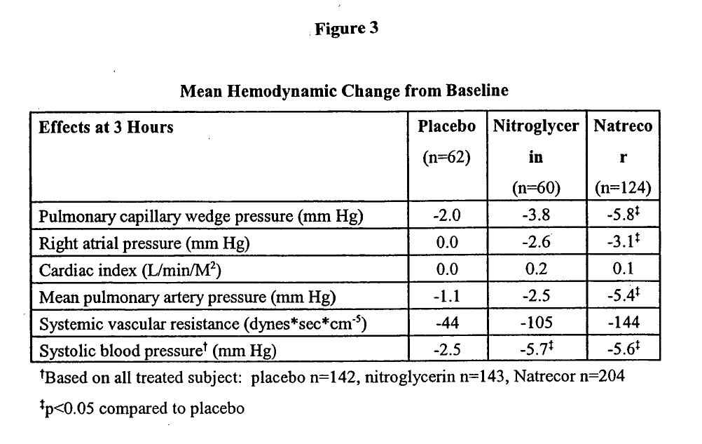

FIG. 2 summarize the changes in the VMAC trial in PCWP and other measures during the first 3 hours. FIG. 3 is the mean hemodynamic change from baseline in patients in the VMAC during the first 3 hours. The VMAC study does not constitute an adequate effectiveness comparison with nitroglycerin. In this trial, the nitroglycerin group provides a rough landmark using a familiar therapy and regimen.

### End on Urine Output

In the VMAC trial, in which the use of diuretics was not restricted, the mean change in volume status (output minus input) during the first 24 hours in the nitroglycerin and Natrecor groups was similar. 1279±1455 mL and 1257±1657 mL, respectively.

### Indications and Usage

Natrecor is indicated for the intravenous treatment of patients with acutely decompensated congestive heart failure who have dyspnea at rest or with minimal activity. In this population the use of Natrecor reduced pulmonary capillary wedge pressure and improved dyspnea.

### Contraindications

Natrecor is contraindicated in patients who are hypersensitive to any of its components. Natrecor should not be used as primary therapy for patients with cardiovenic shock or in patients with a systolic blood pressure <90 mm Hg.

### Warnings

Administration of Natrecor should be avoided in patients; suspected of having, of known to have low cardiac filling pressures.

### Precautions

General: Parenteral administration of protein pharmaceuticals or E. coli derived products should be attended by appropriate precautions in case of an allergic or untoward reaction. No serious allergic or anaphylactic reactions have been reported with Natrecor.

Natrecor is not recommended for patients for whom vasodilating agents arit not appropriate, such as patients with significant valvular stenosis, restrictive or obstructive cardiomyopethy, constrictive pericarditis, pericardial tamponade, or other conditions in which cardiac output is dependent upon venous return, or for patients suspected to have low cardiac filling pressures. (See CONTRAINDICATIONS).

Renal: Natrecor may affect renal function in susceptible individuals. In patients with severe heart failure whose renal function may depend on the activity of the renin-angiotensin-aldosterone system, treatment with Natrecor may be associated with azotemia. When Natrecor was initiated at doses higher than 0.01 μg/kg/min (0.015 and 0.03 μg/kg/min), there was an increased rate of elevated serum creatinine over baseline compared with standard therapies, although the rate of acute renal failure and need for dialysis was not increased. In the 30-day follow-up period in the VMAC trial, 5 patients in the nitroglycerin-group (2%) and 9 patients in the Natrecor group (3%) required first-time dialysis.

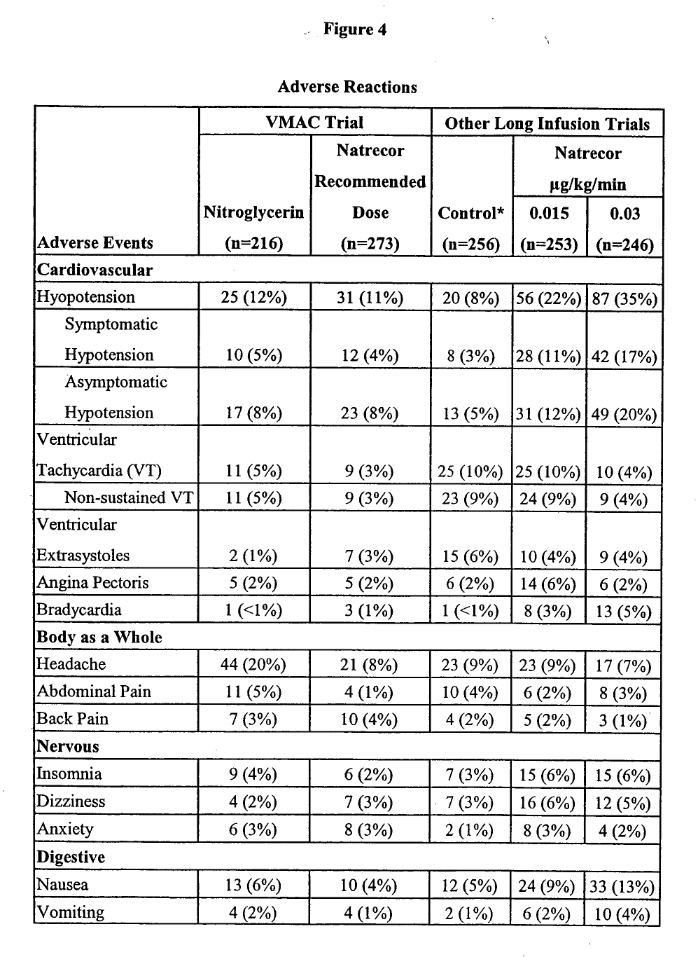

Cardiovascular: Natrecor may cause hypotension. In the VMAC trial, in patients given the recommended dose (2 μg/kg bolus followed by a 0.01 μg/kg/min infusion) or the adjustable dose, the incidence of symptomatic hypotansion in the first 24 hours was similar for Natrecor (4%) and IV nitroglycerin (5%). When hypotension occurred, however, the duration of symptomatic hypotension was longer with Natrecor (mean duration was 2.2 hours) than with nitroglycerin (mean duration was 0.7 hours). In earlier trials, when Narrator was initiated at doses higher than the 2 μg/kg bolus followed by a 0.01 μg/kg/min infusion (i.e., 0.015 and 0.03 μg/kg/min preceded by a small bolus), there wets more hypotensive episodes and these episodes were of greater imengity and duration. They were also more often symptomatic and/or more likely to require medical intervention (see ADVERSE REACTIONS and FIG. 4). Natrecor should be administered only in settings where blood pressure cart be monitored closely, and the dose of Natrecor should be reduced or the drug discontinued in patients who develop hypotension (see Dosing Instructions). The rate of symptomatic hypotension may be increased in patients with a blood pressure <100 mm Hg at baseline, and Natrecor should be used cautiously in these patients. The potential for hypotension may be increased by combining Natrecor with other drugs that may cause hypertension. For example, in the VMAC trial in pedants treated with either Natrecor or nitroglycerin thereapy, the frequency of symptomatic hypotension in patents who received an oral ACE inhibitor was 6%, compared to a frequency of symptomatic hypotension of 1% in patients who did not receive on oral ACE inhibitor.

Drug Interaction: No trials specifically examining potential drug interactions with Natrecor were conducted, although many concomitant drugs were used in clinical trials. No drug interactions were detected except for an increase in symptomatic hypotension in patients receiving oral ACE inhibitors (see PRECAUTIONS, Cardiovascular).

The co-administration of Natrecor with IV vasodilators such as nitroglycerin, nitroprusside, milrinone, or IV ACE inhibitors has not been evaluated (these drugs were not co-administered with Natrecor in clinical trials).

Carcinogenesis, Mutagenesis, Impairment of Fertility: Long-term studies in animals have not been performed to evaluate the carcinogenic potential or the effect on fertility of nesiritide. Nesiritide did not increase the frequency of mutations when used in an in vitro bacterial cell assay (Ames test). No other genotoxicity studies were performed.

Pregnancy: Category C: Animal developmental and reproductive toxicity studies have not been conducted with nasiritide. It is also not known whether Natrecor can cause total harm when administered to pregnant women or can affect reproductive capacity. Natrecor should be used during pregnancy only if the potential benefit justifies any possible risk lo the fetus.

Nursing Mothers: It is not known whether this drug is excreted in human milk. Therefore, caution should be exercised when Natrecor is administered to a nursing woman.

Pediatric Use: The safety and effectiveness of Natrecor in pediatric patents has not been established:

Geriatric Use: Of the total number of subjects in clinical trials treated with Natrecor (n=941), 38% were 65 years or older and 16% were 75 years or older. No overall differences in effectiveness were observed between these subjects and younger subjects, and other reported clinical experience has not identified differences in responses between the elderly and younger patients. Some older individuals may be more sensitive to the effect of Natrecor than younger individuals.

### Adverse Reactions

Adverse events that occurred with at least a 3% frequency during the first 24 hours of Natrecor infusion are shown in Table 6. Adverse events that we not fisted in the above table that occurred in at least 1% of patients who received any of the above Natrecor doses included: Tachycardia, atrial fibrillation, AV mods conduction abnormalities, catheter pain, injection site reaction, confusion, paresthesia, somnolence, tremor, increased cough, hemoptysis, apnea, increased creatinine, sweating, pruritus, rash, leg cramps, amblyopia, anemia. All reported events (at least 1%) are included except time already listed, those too general to be informative, and those not reasonably associated with the use of the drug because they were associated with the condition being treated or are very common in the treated population.

In placebo and active-controlled clinical trials, Natrecor has not been associated with an increase in atrial or ventricular tachyarrhythmias. In placebo-controlled trials, the incidence of VT in both Natrecor and placebo patients was 2%. In the PRECEDENT (Prospective Randomized Evaluation of Cardiac Ectopy with Dobutamine or Natrecor Therapy) trial, the affects of Natrecor (n=163) and dobutamine (n=83) onto provocation or aggravation of existing ventricular arrhythmias in patients with decompensated CHF was compared using Holter monitoring. Treatment with Natrecor (0.015 and 0.03 μg/kg/min without an initial bolus) for 24 hours did not aggravate pre-existing VT or the frequency of premature ventricular beats, compared to a baseline 24-hour Holtor tape.

### Clinical Laboratory

In the PRECEDENT trial, the incidence of elevations in serum creatinine to >0.5 mg/dL above baseline through day 14 was higher in the Natrecor 0.015 μg/kg/min group (17%) and the Natrecor 0.03 μg/kg/min group (19%) than with standard therapy (11%). In the VMAC trial, through day 30, the incidence of elevations in creatinfris to >0.5 mg/dL above baseline was 20% and 21% in the Natrecor (2 μg/kg bolus followed by 0.01 μg/kg/min) and nitroglycerin groups, respectively.

### Effect on Mortality

In the VMAC trial, the mortality rates at six months in the patients receiving Natrecor and nitroglycerin wars 25.1% (95% confidence interval, 20.0% to 30.5%) snul 20.8% (95% confidence interval, 15.5% to 26.5%), respectively. In all controlled trials combined, the mortality rates for Natrecor and active control (including nitroglycerin, dobutamine, nitroprusside, milrinone, amrinone, and dopamine) patients wire 21.5% and 21.7%, respectively.

### Overdosage

No data are available with respect to overdosage in humans. The expected reaction would be excessive hypotension, which should be treated with drug discontinuation or reduction (see PRECAUTIONS) and appropriate measures.

### Dosage and Administration

Natrecor (nesiritide), as currently approved by the Food and Drug Administration (FDA), is for intravenous use only. There is limited experience with administering Natrecor for longer then 48 hours. Blood pressure should be monitored closely during Natrecor administration. If hypotension occurs during the administration of Natrecor, the dose should be reduced or discontinued and other measures to support blood pressure should be started (IV fluids, changes in body position). In the VMAC trial, when symptomatic hypotension occurred, Natrecor was discontinued and subsequently could be restarted at a dose that was reduced by 30% (with no bolus administration) once the patient was stabilized. Because hypotension caused by Natrecor may be prolonged (up to hours), a period of observation may be necessary before restarting the drug.

### Preparation

1. Reconstitute one 1.5 mg vial of Natrecor by adding 5 mL of diluent removed from a pre-filled 250 mL plastic IV bag containing the diluent of choice. The following preservative-free diluents are recommended for reconstitution: 5% Dextrose Injection (D5W), USP; 0.9% Sodium Chloride Injection, USP; 5% Dextrose and 0.45% Sodium Chloride Injection, USP; or 5% Dextrose and 0.2% Sodium Chloride Injection, USP.

2. Do not shake the vial. Rock the vial gently so that all surfaces, including the stopper, are in contact with the diluent to ensure complete reconstitution. Use only a clear, essentially colorless solution.

3. Withdraw the entire contents of the reconstituted Natrecor vial and add to the 250 mL plastic IV bag. This will yield a solution with a concentration of Natrecor of approximately 6 μg/mL. The IV bag should be inverted several times to ensure complete mixing of the solution.

4. Use the reconstituted solution within 24 hours, as Natrecor contains no antimicrobial preservative. Parenteral drug products should be inspected visually for particulate matter and discoloration prior to administration, whenever solution and container permit. Reconstituted vials of Natrecor may be left at Controlled Room Temperature (20-25° C.; 68-77° F.) as per United States Pharmecopeia (USP) or may be refrigerated (2-8° C.; 36-46° F.) for up to 24 hours.

### Dosing Instructions

The recommended dose of Natrecor is an IV bolus of 2 μg/kg followed by a continuous infusion of 0.01 μg/kg/min. Natrecor should not be initiated at a dose that is above the recommended dose.

Prime the IV tubing with an infusion of 25 mL prior to connecting to the patient's vascular access port and prior to administering the bolus or starting the infusion.

Bolus followed by infusion: After preparation of the infusion bag as described previously, withdraw the bolus volume (see table below) from the Natrecor infusion bag, and administer it over approximately 60 seconds through an IV port in the tubing. Immediately following the administration of the bolus, infuse Natrecor at a flow rate of 0.1 mL/kg/hr. This will deliver a Natrecor infusion dose of 0.01 μg/kg/min.

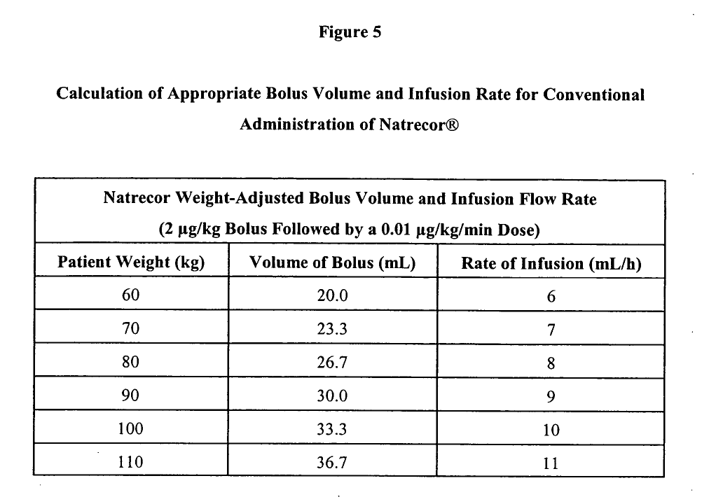

To calculate the appropriate bolus volume and infusion flow rate to deliver a 0.01 μg/kg/min dose, use the following formulas: Bolus Volume (mL) =Patient Weight (kg)÷3; Infusion Flow Rate (mL/hr)=0.1×Patient Weight (kg) (or refer to FIG. 5).

Dose Adjustments: The dose-limiting side effect of Natrecor is hypotension. Do not initiate Natracor at a dose that is higher then the recommended dose of a 2 μg/kg bolus followed by an infusion of 0.0 1 μg/kg/min. In the VMAC trial there was limited experience with increasing the dose of Natrecor above the recommended dose (23 patients, all whom had central hemodynamic monitoring). In those patients, fine infusion dose of Natrecor was increased by 0.005 μg/kg/min (preceded by a bolus of 1 μg/kg), no more frequently than every 3 hours up to a maximum dose of 0.03 μg/kg/min. Natrecor should not be titrated at frequent intervals as is dome with other IV agents that have a shorter half-life.

### Chemical/Physical Interactions

Natrecor is physically and/or chemically incompatible with injectable formulations of heparin, insulin, ethacrynate sodium, bumetanide, enalaprilat, hydralazine, and furosemide. These drugs should not be co-administered as infusions with Natrecor through the same IV catheter. The preservative sodium metabisulfate is incompatible with Natrecor. Injectable drugs that contain sodium metabisulfate should not be administered in the same infusion line as Natrecor. The catheter must be flushed between administration of Natrecor and incompatible drugs. Natrecor binds to heparin and therefore could bind to the heparin lining of heparin-coated catheter, decreasing the amount of Natrecor delivered the patient for some period of time. Therefore, Natrecor must not be administered through a central heparin-coated catheter. Concomitant administration of a heparin infusion through a separate catheter is acceptable.

### Storage

Store Natrecor at controlled room temperature (20-25° C.; 68-77° F.), excursions permitted to 15-30° C. (59-86° F.; see USP Controlled Room Temperature),or refrigerated (2-8° C.; 36-46° F.). Keep in carton until time of use.

### How Supplied

Natrecor is provided as a sterfie lyophilized powder in 1.5 mg, single-use vials. Each carton contains one vial and is available in the following package:

1 vial/carton (NDC 65847-205-25)

U.S. Pat. No. 5,114,923 and 5,674,710.

Distributed by Scios Inc.

820 West Maude Ave

Sunnyvale, Calif. 94085

Copyright 2001 Scios Inc.

NA1030.01

Revised September 2001

Natrecor® is approved for marketing in the U.S. by the Food and Drug Administration and is indicated for the intravenous treatment of patients with acutely decompensated congestive heart failure who have dyspnea at rest or with minimal activity.

### Procedures

### Week 1

Baseline Procedures, All Patients;

Confirm the patient's continued eligibility since the screening visit;

Obtain and record the patient's current weight and vital signs (BP and HR);

Evaluate hydration status (refer to section 7.1.5);

For patients randomized into Group A, determine if weekly inotropic therapy will be utilized;

Perform pregnancy test (if applicable);

Draw blood for baseline plasma BNP levels, chemistry, and hematology samples (see section 6.1);

Draw blood for baseline endothelin-1 and aldosterone (only at selected sites);

Have patient complete the Minnesota Living with Heart Failure (MlwHF) questionnaire;

Request randomization from the study pharmacist or designee;

### Weeks 2-12

All patients, including patients receiving usual long term cardiac medications therapy, are to be evaluated at least once each week during the study period for evaluation even if, in the investigator's judgment, an infusion is not needed at one or more of those visits.

### Weekly Procedures

Prior to Natrecor®, if applicable:

For all patients, regardless of treatment group:

Record patient's vital signs (BP and HR);

Record patient's weight;

Obtain blood for plasma BNP level;

Weekly BUN and creatinine are recommended at each visit to aid in assessing hydration status;

Document all known BUN and creatinine values;

Evaluate patients hydration status (refer to section 7.1.5);

Document cardiac medications taken since last visit;

Query the patients for any adverse events and changes or additions to concomitant medications since the last visit;

Record all medications administered during infusions of Natrecor®.

### Non-Weekly Procedures

For all patients regardless of treatment group:

Obtain Blood samples for endothelin-1 and aldosterone levels at Weeks 4, 8, and 12 prior to any infusions (at selected sites);

BUN and creatinine must be obtained for all patients during Weeks 4, 8, and 12;

Have patient complete the Minnesota Living with Heart Failure Questionnaire at Weeks 4, 8, and 12 (may be done before or during the infusion);

Have patient complete the Patient Global Assessment at Weeks 4, 8, and 12 (before the start of each infusion, if applicable);

Have patient perform a 6-minute walk test at Weeks 6 and 12. The test may be obtained prior to the infusion on the same day or on a day prior to the infusion day, but in the same week;

A Global Assessment is done by the investigator at Weeks 4, 8, and 12 after the infusion.

### Associated Laboratory Assessment

### Laboratory

Blood is drawn for white blood count, hemoglobin and hematocrit at baseline and Week 13. Specimens are analyzed.

Blood is drawn for sodium, potassium, chloride, bicarbonate, blood urea nitrogen (BUN) and creatinine for all patients at baseline and Week 13. Specimens are analyzed.

BUN and creatinine must be obtained for all patients during Week 4, 8, and 12 prior to any infusions (if applicable).

Blood is drawn for plasma B-type natriuretic peptide (BNP), at baseline and prior to Natrecor® infusions at Weeks 4, 8, and 12.

Endothelin-1 and aldosterone levels (at selected sites) at baseline and prior to Natrecor® infusions at Weeks 4, 8, and 12.

Plasma BNP—within 30 minutes of drawing samples, spin the sample in a refrigerated centrifuge for 15 minutes; store plasma in a −70° C. freezer until ready for shipment to Scios Inc. for processing.

Endothelin-1—as soon as possible after drawing, spin in a refrigerated centrifuge for 15 minutes; freeze plasma specimen in a −70° C. freezer until ready for shipment to a central laboratory for processing.

Aldosterone—allow specimen to clot at room temperature for 15-30 minutes, then spin in a refrigerated centrifuge for 15 minutes; store frozen serum in a −70° C. freezer until ready for shipment to a central laboratory for processing.

### Assessments

### New York Heart Association Classification

Assess patients NYHA class using criteria in Table 2 are assessed at screening and Week 13.

### Quality of Life Instruments

The Minnesota Living with Heart Failure (MLwHF) Questionnaire is a validated, self-administered questionnaire comprised of 21 questions which cover signs and symptoms relevant to CHF, physical activity, social interaction, sexual activity, work, and emotions during the previous month. (LeJemtel, T. H., et al., Am. J. Cardiol., 31(2):A83-A (1998)) The MLwHF is administered to the patient at baseline, and at Weeks 4, 8, and 12. The patient may complete the questionnaire prior to or during the infusion.

### 6-Minute Walk Test

To measure functional exercise capacity the patient performs a 6-minute walk test at screening and at Weeks 6 and 12. The test at Week 6 and Week 12 can be obtained prior to the infusion or on a day prior to the infusion day, but in the same week. The walking test is conducted in an enclosed corridor on a course that is approximately 33 m long. The patient is instructed to walk from end to end of the corridor, covering as much distance as they can in the allotted 6-minute time period. (Berry, C., and McMurray, J., Pharmacoeconomics 16(3):247-271 (1999)) The total time walked and the total distance walked is recorded on the CRF. Further information on the 6-minute walk test is included in the Study Guide.

### 2-D Echocardiogram

A two dimensional echocardiogram may be performed on each patient within 28 days after the Week 12 study visit to obtain SVI, LVEDI, and EF measurements.

### Steps for Natrecor® Administration

Step 1: Administer an IV bolus of Natrecor® (obtained from the Natrecor® bag) over approximately 60 seconds directly into the IV catheter through an injection port that is as close as possible to the IV catheter insertion site.

Step 2: Immediately after the Natrecor® bolus is administered, begin the Natrecor® infusion by administering the appropriate Group B or Group C weight-adjusted flow rate. Infusions must be administered via a programmable infusion pump.

The initial Week 1 Natrecor® infusion is started at the initial bolus dose and initial infusion dose rate specified in each Natrecor® treatment arm (Table 1). At the discretion of the investigator, after the initial Natrecor® dose has been tolerated for at least 3 hours, an increased bolus and infusion may be given for the remainder of the treatment (6 hours maximum) for that study visit. At the following visits the Natrecor® dose may be adjusted at the investigator's discretion within a range that is half of the initial Week 1 dose to double the initial Week 1 dose. Those skilled in the art shall adjust dosage such that it is best tolerated by the patient.

### Special Administration Instructions

Natrecor® binds to heparin and therefore could bind to the lining of a heparin-coated catheter, decreasing the amount of Natrecor® study drug delivered to the patient for some period of time. Therefore, Natrecor® must NOT be administered through a heparin-coated catheter. Concomitant administration of a heparin infusion through a separate catheter is permitted.

Natrecor® may be administered through a peripheral or central venous access line but must be infused via a programmable infusion pump. No fluid other than Natrecor® or heparin flush solution should be administered through this line.

### Duration of Natrecor®

All patients randomized to receive Natrecor® are expected to be treated with Natrecor® as the primary IV vasoactive therapy for CHF, and should be treated with Natrecor® for 46 hours weekly for 12 weeks in an infusion clinic or other qualified unit.

### Discontinuation of Natrecor® at the End of Each Infusion

Because of the 18 minute half-life of Natrecor®, it is not necessary to titrate down the dose of Natrecor® before discontinuation. Natrecor® may be discontinued by simply stopping the infusion. Obtain vital signs at 30 minutes and 1 hour after Natrecor® is discontinued.

### Guidelines for Dose Selection at Each Visit

In the Natrecor® treatment arms, the goal is to titrate Natrecor® and continue to treat patients at the maximum dose within the assigned treatment dose range, if tolerated. However, because of fluctuations in hydration status from week to week, adjustments in the dose of study drug may be necessary. Before each infusion is started, the investigator should assess the patient's hydration status and CHF symptoms in order to select the appropriate bolus and infusion for that visit. Natrecor® has vasodilating properties and may lead to increased diuresis and natriuresis in some patients. Repeated infusions of Natrecor® or aggressive diuresis may lead to volume depletion that may necessitate a lower dose of study drug. Alternatively, due to patient's non-compliance with dietary salt restriction or use of diuretics, patients may present with increased volume overload. During weeks when patients are noted to have more severe volume depletion or hypotension, the investigator may consider it appropriate to skip the infusion for that week. Conversely, if patients present with fluid overload, the dose of Natrecor® maybe increased up to the maximum dose within the assigned treatment group.

The evaluation of hydration status or CHF symptoms may include a consideration of the following:

Orthostatic blood pressures;

Systolic blood pressure <90 mm Hg before the start of the infusion;

Changes in laboratory values (e.g., BUN, creatinine, BUN/creatinine ratio) over 1-2 weeks;

Changes in weight over previous 1-2 weeks;

Physical signs and symptoms of volume depletion or volume overload, which may include skin turgor, mucosal hydration, thirst, rales, jugular venous distention, peripheral edema, ascites, hepatomegaly, changes in dyspnea or orthopnea, urine specific gravity, and extra heart sounds;

Recent changes in concomitant medications;

Recent intercurrent illnesses such as gastroenteritis, gastrointestinal bleed, fever or other signs of infections.

### Increasing Natrecor®

If, during a weekly evaluation, the investigator believes that the patient's clinical status is such that the patient may receive a clinical benefit from an increased dose, the dose may be increased up to the maximum allowable infusion dose for that treatment group. The following are guidelines for increasing the Natrecor® infusion if additional clinical effect is desired:

DO NOT increase the Natrecor® dose until the patient has tolerated the lower dose at a previous visit or has received at least 3 hours of the infusion at a lower rate at the current visit.

If, after at least 3 hours of infusion, it is believed that the patient will benefit from a higher dose of Natrecor® and the patient has a SBP=90 mm Hg, the Natrecor® dose may be increased up to the maximum dose level within the patient's treatment group.

To increase the Natrecor® dose, administer the bolus dose that corresponds to the next higher dose level for that treatment group (bolus volume obtained from the Natrecor® IV bag) over approximately 60 seconds, followed by an increase to the next higher infusion rate. For example: If the previous infusion rate was 0.0025 μg/kg/min (preceded by a bolus of 0.5 μg/kg), the patient should receive a Natrecor® bolus of 1 μg/kg followed by an infusion rate of 0.005 μg/kg/min. (See Table 1 for the weight adjusted volume of the IV bolus and the higher infusion flow rate).

Natrecor® may be increased only one time during a weekly infusion.

If the dose of Natrecor® is increased for any reason, monitor BP closely. Record vital signs (BP, HR) at the following times:

Immediately before the dose increase,

At 30 minutes and 1 hour after the dose increase.

Note: The measurements obtained immediately before the dose increase must meet the qualifying criteria for the dose increase (i.e., SBP=90 mm Hg).

### Decreasing Natrecor®

If, prior to a weekly infusion, the patient appears “dry” or the SBP is less than 90 mm Hg, the investigator may opt to decrease the bolus dose and infusion rate of Natrecor® for the current weekly infusion to a dose as low as half of the initial dose administered at Week 1.

Due to Excessive or Rapid Decreases in Blood Pressure. If the patient experiences an excessive or rapid decrease in blood pressure at any time during study drug administration, perform the following procedures:

Discontinue Natrecor® or decrease infusion rate by 50%;

Monitor BP and HR closely until the systolic blood pressure (SBP) stabilizes above 90 mm Hg;

If the infusion has been discontinued, and the SBP has stabilized above 90 mm Hg, Natrecor® infusion may be restarted at a flow rate that is 50% lower than the infusion rate administered before the infusion was stopped, Natrecor® should not be readministered during that day's treatment period);

Intravenous fluids such as normal saline may be administered to treat an episode of hypotension, if appropriate;

After the 50% lower flow rate has been administered for at least 3 hours, if the patient's SBP has stabilized above 90 mm Hg, the Natrecor® infusion rate may be increased. An increase should not be higher than the dose at which hypotension occurred, at that infusion visit.

The previous procedures may be repeated, as clinically appropriate.

If the Natrecor® infusion rate is decreased, or is terminated for any reason, record vital signs (BP, HR) at the following times:

Immediately before the dose decrease, interruption, or termination of Natrecor®,

At 30 minutes and 1 hour, after dose decrease, interruption or discontinuation of the infusion,

If appropriate, record any adverse events.

### Guidelines for Infusion Frequency

Patients may receive infusions as frequently as twice a week or as infrequently as once every other week.

Patients may be more clinically unstable following discharge as they are titrating back onto oral medications and may benefit from more frequent infusions initially. Therefore, the investigator is encouraged to treat patients twice during the first week in order to more rapidly titrate to the best dose for that patient within the specified dose range.

Natrecor® infusions should be administered at least once per week, unless clinically contraindicated. Patients may receive an additional infusion of Natrecor® per week if clinically indicated at any time during the study.

A weekly infusion may be omitted if, in the opinion of the investigator, the patient may not tolerate the infusion for reasons such as systolic blood pressure below 90 mm Hg, dehydration, volume depletion, cardiogenic shock or hospitalization for CHF symptoms. However, in order to maintain compliance with this protocol, infusions must be administered no less than every other week.

Having now fully described the invention, the same will be understood by those with skill in the art that the scope may be performed with a wide and equivalent range of conditions, parameters, and the like, without affecting the spirit or scope of the invention or any embodiment thereof.

TABLE 1
Examples of Useful Doses According to the Invention

| | | Initial | | Minimum | | Maximum | |
|---|---|---|---|---|---|---|---|
| | | | Rate of | Volume of | Rate of | Volume of | Rate of |
| | | Volume of | Initial | Minimum | Minimum | Maximum | Maximum |
| Subject | | Initial Bolus | Infusion | Bolus | Infusion | Bolus | Infusion |
| Weight | | 0.5 μg/kg | 0.0025 μg/kg/min | 0.25 μg/kg | 0.00125 μg/kg/min | 1.0 μg/kg | 0.005 μg/kg/min |
| kg | lb | mL | mL/hr | mL | mL/hr | mL | mL/hr |

Group B Natrecor ® Doses

| 30 | 66 | 2.5 | 0.8 | 1.3 | 0.4 | 5.0 | 1.5 |
|---|---|---|---|---|---|---|---|
| 31 | 68 | 2.6 | 0.8 | 1.3 | 0.4 | 5.2 | 1.6 |
| 32 | 71 | 2.7 | 0.8 | 1.3 | 0.4 | 5.3 | 1.6 |
| 33 | 73 | 2.8 | 0.8 | 1.4 | 0.4 | 5.5 | 1.7 |
| 34 | 75 | 2.8 | 0.9 | 1.4 | 0.4 | 5.7 | 1.7 |
| 35 | 77 | 2.9 | 0.9 | 1.5 | 0.4 | 5.8 | 1.8 |
| 36 | 79 | 3.0 | 0.9 | 1.5 | 0.5 | 6.0 | 1.8 |
| 37 | 82 | 3.1 | 0.9 | 1.5 | 0.5 | 6.2 | 1.9 |
| 38 | 84 | 3.2 | 1.0 | 1.6 | 0.5 | 6.3 | 1.9 |
| 39 | 86 | 3.3 | 1.0 | 1.6 | 0.5 | 6.5 | 2.0 |
| 40 | 88 | 3.3 | 1.0 | 1.7 | 0.5 | 6.7 | 2.0 |
| 41 | 90 | 3.4 | 1.0 | 1.7 | 0.5 | 6.8 | 2.1 |
| 42 | 93 | 3.5 | 1.1 | 1.8 | 0.5 | 7.0 | 2.1 |
| 43 | 95 | 3.6 | 1.1 | 1.8 | 0.5 | 7.2 | 2.2 |
| 44 | 97 | 3.7 | 1.1 | 1.8 | 0.6 | 7.3 | 2.2 |
| 45 | 99 | 3.8 | 1.1 | 1.9 | 0.6 | 7.5 | 2.3 |
| 46 | 101 | 3.8 | 1.2 | 1.9 | 0.6 | 7.7 | 2.3 |
| 47 | 104 | 3.9 | 1.2 | 2.0 | 0.6 | 7.8 | 2.4 |
| 48 | 106 | 4.0 | 1.2 | 2.0 | 0.6 | 8.0 | 2.4 |
| 49 | 108 | 4.1 | 1.2 | 2.0 | 0.6 | 8.2 | 2.5 |
| 50 | 110 | 4.2 | 1.3 | 2.1 | 0.6 | 8.3 | 2.5 |
| 51 | 112 | 4.3 | 1.3 | 2.1 | 0.6 | 8.5 | 2.6 |
| 52 | 115 | 4.3 | 1.3 | 2.2 | 0.7 | 8.7 | 2.6 |
| 53 | 117 | 4.4 | 1.3 | 2.2 | 0.7 | 8.8 | 2.7 |
| 54 | 119 | 4.5 | 1.4 | 2.3 | 0.7 | 9.0 | 2.7 |
| 55 | 121 | 4.6 | 1.4 | 2.3 | 0.7 | 9.2 | 2.8 |
| 56 | 123 | 4.7 | 1.4 | 2.3 | 0.7 | 9.3 | 2.8 |
| 57 | 126 | 4.8 | 1.4 | 2.4 | 0.7 | 9.5 | 2.9 |
| 58 | 128 | 4.8 | 1.5 | 2.4 | 0.7 | 9.7 | 2.9 |
| 59 | 130 | 4.9 | 1.5 | 2.5 | 0.7 | 9.8 | 3.0 |
| 60 | 132 | 5.0 | 1.5 | 2.5 | 0.8 | 10.0 | 3.0 |
| 61 | 135 | 5.1 | 1.5 | 2.5 | 0.8 | 10.2 | 3.1 |
| 62 | 137 | 5.2 | 1.6 | 2.6 | 0.8 | 10.3 | 3.1 |
| 63 | 139 | 5.3 | 1.6 | 2.6 | 0.8 | 10.5 | 3.2 |
| 64 | 141 | 5.3 | 1.6 | 2.7 | 0.8 | 10.7 | 3.2 |
| 65 | 143 | 5.4 | 1.6 | 2.7 | 0.8 | 10.8 | 3.3 |
| 66 | 146 | 5.5 | 1.7 | 2.8 | 0.8 | 11.0 | 3.3 |
| 67 | 148 | 5.6 | 1.7 | 2.8 | 0.8 | 11.2 | 3.4 |
| 68 | 150 | 5.7 | 1.7 | 2.8 | 0.9 | 11.3 | 3.4 |
| 69 | 152 | 5.8 | 1.7 | 2.9 | 0.9 | 11.5 | 3.5 |
| 70 | 154 | 5.8 | 1.8 | 2.9 | 0.9 | 11.7 | 3.5 |
| 71 | 157 | 5.9 | 1.8 | 3.0 | 0.9 | 11.8 | 3.6 |
| 72 | 159 | 6.0 | 1.8 | 3.0 | 0.9 | 12.0 | 3.6 |
| 73 | 161 | 6.1 | 1.8 | 3.0 | 0.9 | 12.2 | 3.7 |
| 74 | 163 | 6.2 | 1.9 | 3.1 | 0.9 | 12.3 | 3.7 |
| 75 | 165 | 6.3 | 1.9 | 3.1 | 0.9 | 12.5 | 3.8 |
| 76 | 168 | 6.3 | 1.9 | 3.2 | 1.0 | 12.7 | 3.8 |
| 77 | 170 | 6.4 | 1.9 | 3.2 | 1.0 | 12.8 | 3.9 |
| 78 | 172 | 6.5 | 2.0 | 3.3 | 1.0 | 13.0 | 3.9 |
| 79 | 174 | 6.6 | 2.0 | 3.3 | 1.0 | 13.2 | 4.0 |
| 80 | 176 | 6.7 | 2.0 | 3.3 | 1.0 | 13.3 | 4.0 |
| 81 | 179 | 6.8 | 2.0 | 3.4 | 1.0 | 13.5 | 4.1 |
| 82 | 181 | 6.8 | 2.1 | 3.4 | 1.0 | 13.7 | 4.1 |
| 83 | 183 | 6.9 | 2.1 | 3.5 | 1.0 | 13.8 | 4.2 |
| 84 | 185 | 7.0 | 2.1 | 3.5 | 1.1 | 14.0 | 4.2 |
| 85 | 187 | 7.1 | 2.1 | 3.5 | 1.1 | 14.2 | 4.3 |
| 86 | 190 | 7.2 | 2.2 | 3.6 | 1.1 | 14.3 | 4.3 |
| 87 | 192 | 7.3 | 2.2 | 3.6 | 1.1 | 14.5 | 4.4 |
| 88 | 194 | 7.3 | 2.2 | 3.7 | 1.1 | 14.7 | 4.4 |
| 89 | 196 | 7.4 | 2.2 | 3.7 | 1.1 | 14.8 | 4.5 |
| 90 | 198 | 7.5 | 2.3 | 3.8 | 1.1 | 15.0 | 4.5 |
| 91 | 201 | 7.6 | 2.3 | 3.8 | 1.1 | 15.2 | 4.6 |
| 92 | 203 | 7.7 | 2.3 | 3.8 | 1.2 | 15.3 | 4.6 |
| 93 | 205 | 7.8 | 2.3 | 3.9 | 1.2 | 15.5 | 4.7 |
| 94 | 207 | 7.8 | 2.4 | 3.9 | 1.2 | 15.7 | 4.7 |
| 95 | 209 | 7.9 | 2.4 | 4.0 | 1.2 | 15.8 | 4.8 |
| 96 | 212 | 8.0 | 2.4 | 4.0 | 1.2 | 16.0 | 4.8 |
| 97 | 214 | 8.1 | 2.4 | 4.0 | 1.2 | 16.2 | 4.9 |
| 98 | 216 | 8.2 | 2.5 | 4.1 | 1.2 | 16.3 | 4.9 |
| 99 | 218 | 8.3 | 2.5 | 4.1 | 1.2 | 16.5 | 5.0 |
| 100 | 221 | 8.3 | 2.5 | 4.2 | 1.3 | 16.7 | 5.0 |
| 101 | 223 | 8.4 | 2.5 | 4.2 | 1.3 | 16.8 | 5.1 |
| 102 | 225 | 8.5 | 2.6 | 4.3 | 1.3 | 17.0 | 5.1 |
| 103 | 227 | 8.6 | 2.6 | 4.3 | 1.3 | 17.2 | 5.2 |
| 104 | 229 | 8.7 | 2.6 | 4.3 | 1.3 | 17.3 | 5.2 |
| 105 | 232 | 8.8 | 2.6 | 4.4 | 1.3 | 17.5 | 5.3 |
| 106 | 234 | 8.8 | 2.7 | 4.4 | 1.3 | 17.7 | 5.3 |
| 107 | 236 | 8.9 | 2.7 | 4.5 | 1.3 | 17.8 | 5.4 |
| 108 | 238 | 9.0 | 2.7 | 4.5 | 1.4 | 18.0 | 5.4 |
| 109 | 240 | 9.1 | 2.7 | 4.5 | 1.4 | 18.2 | 5.5 |
| 110 | 243 | 9.2 | 2.8 | 4.6 | 1.4 | 18.3 | 5.5 |
| 111 | 245 | 9.3 | 2.8 | 4.6 | 1.4 | 18.5 | 5.6 |
| 112 | 247 | 9.3 | 2.8 | 4.7 | 1.4 | 18.7 | 5.6 |
| 113 | 249 | 9.4 | 2.8 | 4.7 | 1.4 | 18.8 | 5.7 |
| 114 | 251 | 9.5 | 2.9 | 4.8 | 1.4 | 19.0 | 5.7 |
| 115 | 254 | 9.6 | 2.9 | 4.8 | 1.4 | 19.2 | 5.8 |
| 116 | 256 | 9.7 | 2.9 | 4.8 | 1.5 | 19.3 | 5.8 |
| 117 | 258 | 9.8 | 2.9 | 4.9 | 1.5 | 19.5 | 5.9 |
| 118 | 260 | 9.8 | 3.0 | 4.9 | 1.5 | 19.7 | 5.9 |
| 119 | 262 | 9.9 | 3.0 | 5.0 | 1.5 | 19.8 | 6.0 |
| 120 | 265 | 10.0 | 3.0 | 5.0 | 1.5 | 20.0 | 6.0 |
| 121 | 267 | 10.1 | 3.0 | 5.0 | 1.5 | 20.2 | 6.1 |
| 122 | 269 | 10.2 | 3.1 | 5.1 | 1.5 | 20.3 | 6.1 |
| 123 | 271 | 10.3 | 3.1 | 5.1 | 1.5 | 20.5 | 6.2 |
| 124 | 273 | 10.3 | 3.1 | 5.2 | 1.6 | 20.7 | 6.2 |
| 125 | 276 | 10.4 | 3.1 | 5.2 | 1.6 | 20.8 | 6.3 |
| 126 | 278 | 10.5 | 3.2 | 5.3 | 1.6 | 21.0 | 6.3 |
| 127 | 280 | 10.6 | 3.2 | 5.3 | 1.6 | 21.2 | 6.4 |
| 128 | 282 | 10.7 | 3.2 | 5.3 | 1.6 | 21.3 | 6.4 |
| 129 | 284 | 10.8 | 3.2 | 5.4 | 1.6 | 21.5 | 6.5 |
| 130 | 287 | 10.8 | 3.3 | 5.4 | 1.6 | 21.7 | 6.5 |
| 131 | 289 | 10.9 | 3.3 | 5.5 | 1.6 | 21.8 | 6.6 |
| 132 | 291 | 11.0 | 3.3 | 5.5 | 1.7 | 22.0 | 6.6 |
| 133 | 293 | 11.1 | 3.3 | 5.5 | 1.7 | 22.2 | 6.7 |
| 134 | 295 | 11.2 | 3.4 | 5.6 | 1.7 | 22.3 | 6.7 |
| 135 | 298 | 11.3 | 3.4 | 5.6 | 1.7 | 22.5 | 6.8 |
| 136 | 300 | 11.3 | 3.4 | 5.7 | 1.7 | 22.7 | 6.8 |
| 137 | 302 | 11.4 | 3.4 | 5.7 | 1.7 | 22.8 | 6.9 |
| 138 | 304 | 11.5 | 3.5 | 5.8 | 1.7 | 23.0 | 6.9 |
| 139 | 306 | 11.6 | 3.5 | 5.8 | 1.7 | 23.2 | 7.0 |
| 140 | 309 | 11.7 | 3.5 | 5.8 | 1.8 | 23.3 | 7.0 |
| 141 | 311 | 11.8 | 3.5 | 5.9 | 1.8 | 23.5 | 7.1 |
| 142 | 313 | 11.8 | 3.6 | 5.9 | 1.8 | 23.7 | 7.1 |
| 143 | 315 | 11.9 | 3.6 | 6.0 | 1.8 | 23.8 | 7.2 |
| 144 | 318 | 12.0 | 3.6 | 6.0 | 1.8 | 24.0 | 7.2 |
| 145 | 320 | 12.1 | 3.6 | 6.0 | 1.8 | 24.2 | 7.3 |
| 146 | 322 | 12.2 | 3.7 | 6.1 | 1.8 | 24.3 | 7.3 |
| 147 | 324 | 12.3 | 3.7 | 6.1 | 1.8 | 24.5 | 7.4 |
| 148 | 326 | 12.3 | 3.7 | 6.2 | 1.9 | 24.7 | 7.4 |
| 149 | 329 | 12.4 | 3.7 | 6.2 | 1.9 | 24.8 | 7.5 |
| 150 | 331 | 12.5 | 3.8 | 6.3 | 1.9 | 25.0 | 7.5 |
| 151 | 333 | 12.6 | 3.8 | 6.3 | 1.9 | 25.2 | 7.6 |
| 152 | 335 | 12.7 | 3.8 | 6.3 | 1.9 | 25.3 | 7.6 |
| 153 | 337 | 12.8 | 3.8 | 6.4 | 1.9 | 25.5 | 7.7 |
| 154 | 340 | 12.8 | 3.9 | 6.4 | 1.9 | 25.7 | 7.7 |
| 155 | 342 | 12.9 | 3.9 | 6.5 | 1.9 | 25.8 | 7.8 |
| 156 | 344 | 13.0 | 3.9 | 6.5 | 2.0 | 26.0 | 7.8 |
| 157 | 346 | 13.1 | 3.9 | 6.5 | 2.0 | 26.2 | 7.9 |
| 158 | 348 | 13.2 | 4.0 | 6.6 | 2.0 | 26.3 | 7.9 |
| 159 | 351 | 13.3 | 4.0 | 6.6 | 2.0 | 26.5 | 8.0 |
| 160 | 353 | 13.3 | 4.0 | 6.7 | 2.0 | 26.7 | 8.0 |
| 161 | 355 | 13.4 | 4.0 | 6.7 | 2.0 | 26.8 | 8.1 |
| 162 | 357 | 13.5 | 4.1 | 6.8 | 2.0 | 27.0 | 8.1 |
| 163 | 359 | 13.6 | 4.1 | 6.8 | 2.0 | 27.2 | 8.2 |
| 164 | 362 | 13.7 | 4.1 | 6.8 | 2.1 | 27.3 | 8.2 |
| 165 | 364 | 13.8 | 4.1 | 6.9 | 2.1 | 27.5 | 8.3 |
| 166 | 366 | 13.8 | 4.2 | 6.9 | 2.1 | 27.7 | 8.3 |
| 167 | 368 | 13.9 | 4.2 | 7.0 | 2.1 | 27.8 | 8.4 |
| 168 | 370 | 14.0 | 4.2 | 7.0 | 2.1 | 28.0 | 8.4 |
| 169 | 373 | 14.1 | 4.2 | 7.0 | 2.1 | 28.2 | 8.5 |
| 170 | 375 | 14.2 | 4.3 | 7.1 | 2.1 | 28.3 | 8.5 |
| 171 | 377 | 14.3 | 4.3 | 7.1 | 2.1 | 28.5 | 8.6 |
| 172 | 379 | 14.3 | 4.3 | 7.2 | 2.2 | 28.7 | 8.6 |
| 173 | 381 | 14.4 | 4.3 | 7.2 | 2.2 | 28.8 | 8.7 |
| 174 | 384 | 14.5 | 4.4 | 7.3 | 2.2 | 29.0 | 8.7 |
| 175 | 386 | 14.6 | 4.4 | 7.3 | 2.2 | 29.2 | 8.8 |
| | | Initial | | Minimum | | Maximum | |
| | | | Rate of | Volume of | Rate of | Volume of | Rate of |
| | | Volume of | Initial | Minimum | Minimum | Maximum | Maximum |
| Subject | | Initial Bolus | Infusion | Bolus | Infusion | Bolus | Infusion |
| Weight | | 1.0 μg/kg | 0.005 μg/kg/min | 0.5 μg/kg | 0.0025 μg/kg/min | 2.0 μg/kg | 0.01 μg/kg/min |
| kg | lb | mL | mL/hr | mL | mL/hr | mL | mL/hr |

Group C Natrecor ® Doses

| | | | | | | | |
|---|---|---|---|---|---|---|---|
| 30 | 66 | 5.0 | 1.5 | 2.5 | 0.8 | 10.0 | 3.0 |
| 31 | 68 | 5.2 | 1.6 | 2.6 | 0.8 | 10.3 | 3.1 |
| 32 | 71 | 5.3 | 1.6 | 2.7 | 0.8 | 10.7 | 3.2 |
| 33 | 73 | 5.5 | 1.7 | 2.8 | 0.8 | 11.0 | 3.3 |
| 34 | 75 | 5.7 | 1.7 | 2.8 | 0.9 | 11.3 | 3.4 |
| 35 | 77 | 5.8 | 1.8 | 2.9 | 0.9 | 11.7 | 3.5 |
| 36 | 79 | 6.0 | 1.8 | 3.0 | 0.9 | 12.0 | 3.6 |
| 37 | 82 | 6.2 | 1.9 | 3.1 | 0.9 | 12.3 | 3.7 |
| 38 | 84 | 6.3 | 1.9 | 3.2 | 1.0 | 12.7 | 3.8 |
| 39 | 86 | 6.5 | 2.0 | 3.3 | 1.0 | 13.0 | 3.9 |
| 40 | 88 | 6.7 | 2.0 | 3.3 | 1.0 | 13.3 | 4.0 |
| 41 | 90 | 6.8 | 2.1 | 3.4 | 1.0 | 13.7 | 4.1 |
| 42 | 93 | 7.0 | 2.1 | 3.5 | 1.1 | 14.0 | 4.2 |
| 43 | 95 | 7.2 | 2.2 | 3.6 | 1.1 | 14.3 | 4.3 |
| 44 | 97 | 7.3 | 2.2 | 3.7 | 1.1 | 14.7 | 4.4 |
| 45 | 99 | 7.5 | 2.3 | 3.8 | 1.1 | 15.0 | 4.5 |
| 46 | 101 | 7.7 | 2.3 | 3.8 | 1.2 | 15.3 | 4.6 |
| 47 | 104 | 7.8 | 2.4 | 3.9 | 1.2 | 15.7 | 4.7 |
| 48 | 106 | 8.0 | 2.4 | 4.0 | 1.2 | 16.0 | 4.8 |
| 49 | 108 | 8.2 | 2.5 | 4.1 | 1.2 | 16.3 | 4.9 |
| 50 | 110 | 8.3 | 2.5 | 4.2 | 1.3 | 16.7 | 5.0 |
| 51 | 112 | 8.5 | 2.6 | 4.3 | 1.3 | 17.0 | 5.1 |
| 52 | 115 | 8.7 | 2.6 | 4.3 | 1.3 | 17.3 | 5.2 |
| 53 | 117 | 8.8 | 2.7 | 4.4 | 1.3 | 17.7 | 5.3 |
| 54 | 119 | 9.0 | 2.7 | 4.5 | 1.4 | 18.0 | 5.4 |
| 55 | 121 | 9.2 | 2.8 | 4.6 | 1.4 | 18.3 | 5.5 |
| 56 | 123 | 9.3 | 2.8 | 4.7 | 1.4 | 18.7 | 5.6 |
| 57 | 126 | 9.5 | 2.9 | 4.8 | 1.4 | 19.0 | 5.7 |
| 58 | 128 | 9.7 | 2.9 | 4.8 | 1.5 | 19.3 | 5.8 |
| 59 | 130 | 9.8 | 3.0 | 4.9 | 1.5 | 19.7 | 5.9 |
| 60 | 132 | 10.0 | 3.0 | 5.0 | 1.5 | 20.0 | 6.0 |
| 61 | 135 | 10.2 | 3.1 | 5.1 | 1.5 | 20.3 | 6.1 |
| 62 | 137 | 10.3 | 3.1 | 5.2 | 1.6 | 20.7 | 6.2 |
| 63 | 139 | 10.5 | 3.2 | 5.3 | 1.6 | 21.0 | 6.3 |
| 64 | 141 | 10.7 | 3.2 | 5.3 | 1.6 | 21.3 | 6.4 |
| 65 | 143 | 10.8 | 3.3 | 5.4 | 1.6 | 21.7 | 6.5 |
| 66 | 146 | 11.0 | 3.3 | 5.5 | 1.7 | 22.0 | 6.6 |
| 67 | 148 | 11.2 | 3.4 | 5.6 | 1.7 | 22.3 | 6.7 |
| 68 | 150 | 11.3 | 3.4 | 5.7 | 1.7 | 22.7 | 6.8 |
| 69 | 152 | 11.5 | 3.5 | 5.8 | 1.7 | 23.0 | 6.9 |
| 70 | 154 | 11.7 | 3.5 | 5.8 | 1.8 | 23.3 | 7.0 |
| 71 | 157 | 11.8 | 3.6 | 5.9 | 1.8 | 23.7 | 7.1 |
| 72 | 159 | 12.0 | 3.6 | 6.0 | 1.8 | 24.0 | 7.2 |
| 73 | 161 | 12.2 | 3.7 | 6.1 | 1.8 | 24.3 | 7.3 |
| 74 | 163 | 12.3 | 3.7 | 6.2 | 1.9 | 24.7 | 7.4 |
| 75 | 165 | 12.5 | 3.8 | 6.3 | 1.9 | 25.0 | 7.5 |
| 76 | 168 | 12.7 | 3.8 | 6.3 | 1.9 | 25.3 | 7.6 |
| 77 | 170 | 12.8 | 3.9 | 6.4 | 1.9 | 25.7 | 7.7 |
| 78 | 172 | 13.0 | 3.9 | 6.5 | 2.0 | 26.0 | 7.8 |
| 79 | 174 | 13.2 | 4.0 | 6.6 | 2.0 | 26.3 | 7.9 |
| 80 | 176 | 13.3 | 4.0 | 6.7 | 2.0 | 26.7 | 8.0 |
| 81 | 179 | 13.5 | 4.1 | 6.8 | 2.0 | 27.0 | 8.1 |
| 82 | 181 | 13.7 | 4.1 | 6.8 | 2.1 | 27.3 | 8.2 |
| 83 | 183 | 13.8 | 4.2 | 6.9 | 2.1 | 27.7 | 8.3 |
| 84 | 185 | 14.0 | 4.2 | 7.0 | 2.1 | 28.0 | 8.4 |
| 85 | 187 | 14.2 | 4.3 | 7.1 | 2.1 | 28.3 | 8.5 |
| 86 | 190 | 14.3 | 4.3 | 7.2 | 2.2 | 28.7 | 8.6 |
| 87 | 192 | 14.5 | 4.4 | 7.3 | 2.2 | 29.0 | 8.7 |
| 88 | 194 | 14.7 | 4.4 | 7.3 | 2.2 | 29.3 | 8.8 |
| 89 | 196 | 14.8 | 4.5 | 7.4 | 2.2 | 29.7 | 8.9 |
| 90 | 198 | 15.0 | 4.5 | 7.5 | 2.3 | 30.0 | 9.0 |
| 91 | 201 | 15.2 | 4.6 | 7.6 | 2.3 | 30.3 | 9.1 |
| 92 | 203 | 15.3 | 4.6 | 7.7 | 2.3 | 30.7 | 9.2 |
| 93 | 205 | 15.5 | 4.7 | 7.8 | 2.3 | 31.0 | 9.3 |
| 94 | 207 | 15.7 | 4.7 | 7.8 | 2.4 | 31.3 | 9.4 |
| 95 | 209 | 15.8 | 4.8 | 7.9 | 2.4 | 31.7 | 9.5 |
| 96 | 212 | 16.0 | 4.8 | 8.0 | 2.4 | 32.0 | 9.6 |
| 97 | 214 | 16.2 | 4.9 | 8.1 | 2.4 | 32.3 | 9.7 |
| 98 | 216 | 16.3 | 4.9 | 8.2 | 2.5 | 32.7 | 9.8 |
| 99 | 218 | 16.5 | 5.0 | 8.3 | 2.5 | 33.0 | 9.9 |
| 100 | 221 | 16.7 | 5.0 | 8.3 | 2.5 | 33.3 | 10.0 |
| 101 | 223 | 16.8 | 5.1 | 8.4 | 2.5 | 33.7 | 10.1 |
| 102 | 225 | 17.0 | 5.1 | 8.5 | 2.6 | 34.0 | 10.2 |
| 103 | 227 | 17.2 | 5.2 | 8.6 | 2.6 | 34.3 | 10.3 |
| 104 | 229 | 17.3 | 5.2 | 8.7 | 2.6 | 34.7 | 10.4 |
| 105 | 232 | 17.5 | 5.3 | 8.8 | 2.6 | 35.0 | 10.5 |
| 106 | 234 | 17.7 | 5.3 | 8.8 | 2.7 | 35.3 | 10.6 |
| 107 | 236 | 17.8 | 5.4 | 8.9 | 2.7 | 35.7 | 10.7 |
| 108 | 238 | 18.0 | 5.4 | 9.0 | 2.7 | 36.0 | 10.8 |
| 109 | 240 | 18.2 | 5.5 | 9.1 | 2.7 | 36.3 | 10.9 |
| 110 | 243 | 18.3 | 5.5 | 9.2 | 2.8 | 36.7 | 11.0 |
| 111 | 245 | 18.5 | 5.6 | 9.3 | 2.8 | 37.0 | 11.1 |
| 112 | 247 | 18.7 | 5.6 | 9.3 | 2.8 | 37.3 | 11.2 |
| 113 | 249 | 18.8 | 5.7 | 9.4 | 2.8 | 37.7 | 11.3 |
| 114 | 251 | 19.0 | 5.7 | 9.5 | 2.9 | 38.0 | 11.4 |
| 115 | 254 | 19.2 | 5.8 | 9.6 | 2.9 | 38.3 | 11.5 |
| 116 | 256 | 19.3 | 5.8 | 9.7 | 2.9 | 38.7 | 11.6 |
| 117 | 258 | 19.5 | 5.9 | 9.8 | 2.9 | 39.0 | 11.7 |
| 118 | 260 | 19.7 | 5.9 | 9.8 | 3.0 | 39.3 | 11.8 |
| 119 | 262 | 19.8 | 6.0 | 9.9 | 3.0 | 39.7 | 11.9 |
| 120 | 265 | 20.0 | 6.0 | 10.0 | 3.0 | 40.0 | 12.0 |
| 121 | 267 | 20.2 | 6.1 | 10.1 | 3.0 | 40.3 | 12.1 |
| 122 | 269 | 20.3 | 6.1 | 10.2 | 3.1 | 40.7 | 12.2 |
| 123 | 271 | 20.5 | 6.2 | 10.3 | 3.1 | 41.0 | 12.3 |
| 124 | 273 | 20.7 | 6.2 | 10.3 | 3.1 | 41.3 | 12.4 |
| 125 | 276 | 20.8 | 6.3 | 10.4 | 3.1 | 41.7 | 12.5 |
| 126 | 278 | 21.0 | 6.3 | 10.5 | 3.2 | 42.0 | 12.6 |
| 127 | 280 | 21.2 | 6.4 | 10.6 | 3.2 | 42.3 | 12.7 |
| 128 | 282 | 21.3 | 6.4 | 10.7 | 3.2 | 42.7 | 12.8 |
| 129 | 284 | 21.5 | 6.5 | 10.8 | 3.2 | 43.0 | 12.9 |
| 130 | 287 | 21.7 | 6.5 | 10.8 | 3.3 | 43.3 | 13.0 |
| 131 | 289 | 21.8 | 6.6 | 10.9 | 3.3 | 43.7 | 13.1 |
| 132 | 291 | 22.0 | 6.6 | 11.0 | 3.3 | 44.0 | 13.2 |
| 133 | 293 | 22.2 | 6.7 | 11.1 | 3.3 | 44.3 | 13.3 |
| 134 | 295 | 22.3 | 6.7 | 11.2 | 3.4 | 44.7 | 13.4 |
| 135 | 298 | 22.5 | 6.8 | 11.3 | 3.4 | 45.0 | 13.5 |
| 136 | 300 | 22.7 | 6.8 | 11.3 | 3.4 | 45.3 | 13.6 |
| 137 | 302 | 22.8 | 6.9 | 11.4 | 3.4 | 45.7 | 13.7 |
| 138 | 304 | 23.0 | 6.9 | 11.5 | 3.5 | 46.0 | 13.8 |
| 139 | 306 | 23.2 | 7.0 | 11.6 | 3.5 | 46.3 | 13.9 |
| 140 | 309 | 23.3 | 7.0 | 11.7 | 3.5 | 46.7 | 14.0 |
| 141 | 311 | 23.5 | 7.1 | 11.8 | 3.5 | 47.0 | 14.1 |
| 142 | 313 | 23.7 | 7.1 | 11.8 | 3.6 | 47.3 | 14.2 |
| 143 | 315 | 23.8 | 7.2 | 11.9 | 3.6 | 47.7 | 14.3 |
| 144 | 318 | 24.0 | 7.2 | 12.0 | 3.6 | 48.0 | 14.4 |
| 145 | 320 | 24.2 | 7.3 | 12.1 | 3.6 | 48.3 | 14.5 |
| 146 | 322 | 24.3 | 7.3 | 12.2 | 3.7 | 48.7 | 14.6 |
| 147 | 324 | 24.5 | 7.4 | 12.3 | 3.7 | 49.0 | 14.7 |
| 148 | 326 | 24.7 | 7.4 | 12.3 | 3.7 | 49.3 | 14.8 |
| 149 | 329 | 24.8 | 7.5 | 12.4 | 3.7 | 49.7 | 14.9 |
| 150 | 331 | 25.0 | 7.5 | 12.5 | 3.8 | 50.0 | 15.0 |
| 151 | 333 | 25.2 | 7.6 | 12.6 | 3.8 | 50.3 | 15.1 |
| 152 | 335 | 25.3 | 7.6 | 12.7 | 3.8 | 50.7 | 15.2 |
| 153 | 337 | 25.5 | 7.7 | 12.8 | 3.8 | 51.0 | 15.3 |
| 154 | 340 | 25.7 | 7.7 | 12.8 | 3.9 | 51.3 | 15.4 |
| 155 | 342 | 25.8 | 7.8 | 12.9 | 3.9 | 51.7 | 15.5 |
| 156 | 344 | 26.0 | 7.8 | 13.0 | 3.9 | 52.0 | 15.6 |
| 157 | 346 | 26.2 | 7.9 | 13.1 | 3.9 | 52.3 | 15.7 |
| 158 | 348 | 26.3 | 7.9 | 13.2 | 4.0 | 52.7 | 15.8 |
| 159 | 351 | 26.5 | 8.0 | 13.3 | 4.0 | 53.0 | 15.9 |
| 160 | 353 | 26.7 | 8.0 | 13.3 | 4.0 | 53.3 | 16.0 |
| 161 | 355 | 26.8 | 8.1 | 13.4 | 4.0 | 53.7 | 16.1 |
| 162 | 357 | 27.0 | 8.1 | 13.5 | 4.1 | 54.0 | 16.2 |
| 163 | 359 | 27.2 | 8.2 | 13.6 | 4.1 | 54.3 | 16.3 |
| 164 | 362 | 27.3 | 8.2 | 13.7 | 4.1 | 54.7 | 16.4 |
| 165 | 364 | 27.5 | 8.3 | 13.8 | 4.1 | 55.0 | 16.5 |
| 166 | 366 | 27.7 | 8.3 | 13.8 | 4.2 | 55.3 | 16.6 |
| 167 | 368 | 27.8 | 8.4 | 13.9 | 4.2 | 55.7 | 16.7 |
| 168 | 370 | 28.0 | 8.4 | 14.0 | 4.2 | 56.0 | 16.8 |
| 169 | 373 | 28.2 | 8.5 | 14.1 | 4.2 | 56.3 | 16.9 |
| 170 | 375 | 28.3 | 8.5 | 14.2 | 4.3 | 56.7 | 17.0 |
| 171 | 377 | 28.5 | 8.6 | 14.3 | 4.3 | 57.0 | 17.1 |
| 172 | 379 | 28.7 | 8.6 | 14.3 | 4.3 | 57.3 | 17.2 |
| 173 | 381 | 28.8 | 8.7 | 14.4 | 4.3 | 57.7 | 17.3 |
| 174 | 384 | 29.0 | 8.7 | 14.5 | 4.4 | 58.0 | 17.4 |
| 175 | 386 | 29.2 | 8.8 | 14.6 | 4.4 | 58.3 | 17.5 |

TABLE 2
New York Heart Association Functional Classification

| | |
|---|---|
| 1. | Patients with cardiac disease but without resulting limitations of |
| | physical activity. Ordinary physical activity does not cause undue |
| | fatigue, palpitation, dyspnea, or anginal pain. |
| 2. | Patients with cardiac disease resulting in slight limitation of |
| | physical activity. They are comfortable at rest. Ordinary physical |
| | activity results in fatigue, palpitation, dyspnea, or anginal pain. |
| 3. | Patients with cardiac disease resulting in marked limitation of |
| | physical activity. They are comfortable at rest. Less than ordinary |
| | physical activity causes fatigue, palpitation, dyspnea, or anginal pain. |
| 4. | Patients with cardiac disease resulting in inability to carry on any |
| | physical activity without discomfort. Symptoms of cardiac |
| | insufficiency or of the anginal syndrome may be present even at rest. |
| | If any physical activity is undertaken, discomfort is increased. |

What is claimed is:

1. A method for treating congestive heart failure in a mammal comprising administering a therapeutically effective dose of a natriuretic peptide to said mammal.

2. The method of claim 1, wherein said dose is an adjunct to oral therapy.

3. The method of claim 1, wherein the said mammal is in a compensated state of congestive heart failure.

4. The method of claim 1, wherein the said mammal is in a decompensated state of congestive heart failure.

5. The method of claim 1, wherein said natriuretic peptide is selected from the group consisting of an atrial natriuretic peptide (ANP), a B-type natriuretic peptide (BNP) and a C-type natriuretic peptide (CNP).

6. The method of claim 5, wherein the said natriuretic peptide is B-type natriuretic peptide.

7. The method of claim 6, wherein said B-type natriuretic peptide (BNP) is selected from the group consisting of human BNP, pig BNP, rat BNP, and chicken BNP.

8. The method of claim 6, wherein said natriuretic peptide is a human B-type natriuretic peptide.

9. The method of claim 6, wherein said natriuretic peptide is a recombinant B-type natriuretic peptide.

10. The method of claim 5, wherein said natriuretic peptide is atrial natriuretic peptide (ANP).

11. The method of claim 10, wherein said atrial natriuretic peptide (ANP) is selected from the group consisting of frog ANP and human ANP.

12. The method of claim 10, wherein said atrial natriuretic peptide (ANP) is human ANP.

13. The method of claim 10, wherein said atrial natriuretic peptide (ANP) is recombinant ANP.

14. The method of claim 5, wherein said natriuretic peptide is C-type natriuretic peptide (CNP).

15. The method of claim 14, wherein said C-type natriuretic peptide (CNP) is selected from the group consisting of chicken CNP, rat CNP and human CNP.

16. The method of claim 15, wherein said C-type natriuretic peptide (CNP) is a human C-type natriuretic peptide.

17. The method of claim 15, wherein said natriuretic peptide is a recombinant C-type natriuretic peptide.

18. The method of claim 1, wherein said natriuretic peptide is administered by bolus, infusion or a combination of bolus and infusion.

19. The method of claim 18, wherein said natriuretic peptide is administered by bolus.

20. The method of claim 18, wherein said natriuretic peptide is administered by infusion.

21. The method of claim 18, wherein said natriuretic peptide is administered by a combination of bolus and infusion.

22. The method of claim 18, wherein said natriuretic peptide is administered by bolus and intermittent infusion.

23. The method of claim 20 and 21, wherein said infusion is intermittant.

24. The method of claim 20 and 21, wherein said infusion is serial.

25. The method of claim 20 and 21, wherein said infusion is continuous.

26. The method of claim 19, wherein said natriuretic peptide is administered by bolus at a dose of not less than 2 μg/kg body weight.

27. The method of claim 24, wherein said natriuretic peptide is administered by serial infusion at a dose less than 0.01 μg/kg body weight.

28. The method of claim 23, wherein said natriuretic peptide is administered by intermittent infusion at a dose less than 0.01 μg/kg body weight.

29. The method of claim 1, wherein said natriuretic peptide is administered by intravenous, intramuscular, subcutaneous, intradermal, intrasternal, intraperitoneal or intra-articular injection.

30. The method of claim 1, wherein the said natriuretic peptide is administered prophylactically to said mammal.

31. The method of claim 1, wherein said natriuretic peptide is administered to said mammal for 10 hours.

32. The method of claim 16, wherein said natriuretic peptide is administered to said mammal for 4-6 hours weekly.

33. The method of claim 1, wherein said natriuretic peptide is administered to said mammal at an infusion rate of 0.00125 μg/kg body weight/min to 0.01 μg/kg body weight/min.

34. The method of claim 19, wherein said natriuretic peptide is administered to said at 0.005 μg/kg body weight/min.

35. The method of claim 1, wherein said therapeutically effective dose comprises 0.25 μg/kg body weight to 1.75 μg/kg body weight of said natriuretic peptide.

36. A kit for the treatment of congestive heart failure by administration of a therapeutically effective dose of a natriuretic peptide, wherein the natriuretic peptide is packaged separately.

37. The kit as claimed in claim 36, wherein said natriuretic peptide is selected from the group consisting of an atrial natriuretic peptide (ANP), a B-type natriuretic peptide (BNP) and a C-type natriuretic peptide (CNP).

38. The kit as claimed in claim 37, wherein said natriuretic peptide is a humane B-type natriuretic peptide.

39. The kit as claimed in claim 37, wherein said natriuretic peptide is a recombinant human B-type natriuretic peptide.
